(19) United States
(12) Patent Application Publication
(10) Pub. No.: US 2007/0048756 A1
(43) Pub. Date: Mar. 1, 2007

(54) Methods for whole genome association studies

(75) Inventors: Rui Mei, Santa Clara, CA (US); Sean Walsh, Danville, CA (US); Hajime Matsuzaki, Palo Alto, CA (US); Michael H. Shapero, Redwood City, CA (US); Keith W. Jones, Sunnyvale, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/406,880

(22) Filed: Apr. 18, 2006

Related U.S. Application Data
(60) Provisional application No. 60/672,744, filed on Apr. 18, 2005.
(60) Provisional application No. 60/690,308, filed on Jun. 13, 2005.

(51) Int. Cl. C12Q 1/68; G06F 19/00

(52) U.S. Cl. 435/6; 702/20

(57) ABSTRACT

Methods for determining the genotype of more than 400,000 Single Nucleotide Polymorphisms (SNPs) in samples of genomic DNA are provided. A collection of SNPs that may be interrogated by the methods is disclosed in SEQ ID NO: 1-1,074,930. Each sequence is the sequence of a human SNP allele and the 16 bases flanking the SNP on either side. A sequence for each allele is included. In some aspects arrays of probes to interrogate the genotype of a collection of SNPs are disclosed. In preferred aspects the probes are 17 or more contiguous nucleotides from a sequence in SEQ ID NO: 1-1,074,930 or its complement.

### RELATED APPLICATIONS

This application is related to U.S. Provisional Application No. 60/672,744, filed Apr. 18, 2005 and 60/690,308 filed Jun. 13, 2005. The entire teachings of the above applications are incorporated herein by reference in their entirety for all purposes.

### FIELD OF THE INVENTION

The methods and kits of the invention relate generally to genotyping greater than 500,000 Single Nucleotide Polymorphisms (SNPs) in samples of genomic DNA.

### REFERENCE TO SEQUENCE LISTING

The Sequence Listing submitted on compact disc is hereby incorporated by reference. The machine format for the discs is IBM-PC, the operating system compatibility is MS-WINDOWS XP, the file on the disc is titled “3732.2seqlist.txt”, the file is 158 MB and the compact discs were created on Apr. 18, 2006.

### BACKGROUND OF THE INVENTION

Single nucleotide polymorphisms (SNPs) have emerged as the marker of choice for genome wide association studies and genetic linkage studies. Building SNP maps of the genome will provide the framework for new studies to identify the underlying genetic basis of complex diseases such as cancer, mental illness and diabetes. Identification of the genetic polymorphisms that contribute to susceptibility for common diseases will facilitate the development of diagnostics and therapeutics, see Carlson et al., Nature 429:446-452 (2004). Whole-genome association studies may be used to identify polymorphisms with disease associations. These studies require the analysis of much denser panels of markers than are required for linkage analysis in families and benefit from technologies that facilitate the analysis of hundreds of thousands of polymorphisms, see, The International HapMap Consortium, Nature 426, 789-796 (2003).

### SUMMARY OF INVENTION

A method for detection of greater than about 500,000 Single Nucleotide Polymorphisms (SNPs) in samples of genomic DNA is disclosed. The methods include a method for amplifying genomic DNA sequences after fragmentation with a selected restriction enzyme, ligation to adaptors, dilution of the DNA fragments, and amplification using one or a few common primers. The amplified fragments are purified, labeled, for example, using fluorescent or chemiluminescent labels, hybridized to an array of probes, washed, and stained. Hybridization patterns are analyzed using computer systems and genotypes that are characteristic of the sample are detected. The genotype information may be used for example, in studies of whole genome association, for example for disease or drug response, linkage studies, and copy number analysis.

In one aspect the more than 500,000 SNPs represented by the sequence listing are useful for whole genome association studies over a variety of populations.

In on aspect the polymorphisms are interrogated by hybridization to an array of probes, wherein each probe is at least 17, 18-21, 21-25 or 26-33 consecutive bases from one sequence selected from SEQ ID NO: 1-1,074,930 or the complements of SEQ ID NO: 1-1,074,930.

In one aspect a collection or probes for interrogating the genotype of a plurality of SNPs is disclosed. The sequence listing includes each allele of the SNPs to be interrogated with 16 bases flanking the polymorphic position on either side. Arrays of probes can be designed using the sequences in the sequence listing. Probes may be 17, 18, 19, 20, 21, 22, 23, 24, 25 or 26-33 bases in length.

Kits including adaptor sequences, primers, ligase, restriction enzymes, DNA polymerase, which may be thermal stable, dNTPs, DNA labeling reagent, terminal deoxytransferase and buffers, which may include additives such as Betaine or DMSO, may be provided. The kits may include an array for interrogating the genotype of a plurality of SNPs.

### DETAILED DESCRIPTION OF THE PREFERRED EMBODIMENTS

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes. See also, Fodor et al., Science 251(4995), 767-73, 1991, Fodor et al., Nature 364(6437), 555-6, 1993 and Pease et al. PNAS USA 91(11), 5022-6, 1994 for methods of synthesizing and using microarrays.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GENECHIP. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Additional methods of genotyping, complexity reduction and nucleic acid amplification are disclosed in U.S. Patent Application Nos. 60/508,418, 60/468,925, 60/493,085, 09/920,491, 10/442,021, 10/654,281, 10/316,811, 10/646,674, 10/272,155, 10/681,773, 10/712,616, 10/880,143, 10/891,260 and 10/918,501 and U.S. Pat. No. 6,582,938. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. Modifications to PCR may also be used, for example, the inclusion of Betaine or trimethylglycine, which has been disclosed, for example, in Rees et al. Biochemistry 32:137-144 (1993), and in U.S. Pat. Nos. 6,270,962 and 5,545,539. The sample may be amplified on the array. See, for example, U.S. Pat. No. 6,300,070.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491, 09/910,292, and 10/013,598.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909, 5,861,245), nucleic acid based sequence amplification (NABSA), rolling circle amplification (RCA), multiple displacement amplification (MDA) (U.S. Pat. Nos. 6,124,120 and 6,323,009) and circle-to-circle amplification (C2CA) (Dahl et al. Proc. Natl. Acad. Sci 101:4548-4553 (2004). Other amplification methods that may be used are described in, U.S. Pat. Nos. 5,242,794, 5,494,810, 5,409,818, 4,988,617, 6,063,603 and 5,554,517 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), U.S. Ser. No. 09/910,292 (U.S. Patent Application Publication 20030082543), and U.S. Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The whole genome sampling assay (WGSA) is described, for example in Kennedy et al., Nat. Biotech. 21, 1233-1237 (2003), Matsuzaki et al., Gen. Res. 14: 414-425, (2004), and Matsuzaki, et al. Nature Methods 1:109-111 (2004). Algorithms for use with mapping assays are described, for example, in Liu et al., Bioinformatics 19: 2397-2403 (2003) and Di et al. Bioinformatics 21:1958 (2005). Additional methods related to WGSA and arrays useful for WGSA and applications of WGSA are disclosed, for example, in U.S. Patent Application No. 60/676,058 filed 4/29/2005, 60/616,273 filed Oct. 5, 2004, U.S. Ser. No. 10/912,445, 11/044,831, 10/442,021, 10/650,332 and 10/463,991. Genome wide association studies using mapping assays are described in, for example, Hu et al., Cancer Res.;65(7):2542-6 (2005), Mitra et al., Cancer Res., 64(21):8116-25 (2004), Butcher et al., Hum Mol Genet., 14(10):1315-25 (2005), and Klein et al., Science, 308(5720):385-9 (2005). Each of these references is incorporated herein by reference in its entirety for all purposes.

Additionally, the present invention may have preferred embodiments that include methods for providing genetic information over networks such as the Internet as shown in U.S. Ser. No. 10/063,559 (United States Publication No. US20020183936), 60/349,546, 60/376,003, 60/394,574 and 60/403,381.

The term “adaptor” refers to an oligonucleotide of at least 5, 10, or 15 bases and preferably no more than 100 to 200 bases in length and more preferably no more than 50 to 60 bases in length, that may be attached to the end of a nucleic acid. Adaptor sequences may be synthesized using any methods known to those of skill in the art. For the purposes of this invention they may comprise, for example, priming sites, the complement of a priming site, recognition sites for endonucleases, common sequences and promoters. The adaptor may be entirely or substantially double stranded. A double stranded adaptor may comprise two oligonucleotides that are at least partially complementary. The adaptor may be phosphorylated or unphosphorylated on one or both strands. Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with PvuII the blunt ends can be converted to a two base pair overhang by incubating the fragments with Klenow in the presence of dTTP and dCTP. Overhangs may also be converted to blunt ends by filling in an overhang or removing an overhang.

In many aspects adaptors may be ligated to restriction fragments. Methods of ligation will be known to those of skill in the art and are described, for example, in Sambrook et at. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′ to 5′ phosphodiester bond, substrates include single-stranded RNA and DNA as well as dinucleoside pyrophosphates; or any other methods described in the art. Different enzymes generate different overhangs and the overhang of the adaptor can be targeted to ligated to fragments generated by selected restriction enzymes.

In some embodiments a double stranded adaptor is used and only one strand is ligated to the fragments. Ligation of one strand of an adaptor may be selectively blocked. Any known method to block ligation of one strand may be employed. For example, one strand of the adaptor can be designed to introduce a gap of one or more nucleotides between the 5′ end of that strand of the adaptor and the 3′ end of the target nucleic acid. Absence of a phosphate from the 5′ end of an adaptor will block ligation of that 5′ end to an available 3′OH. For additional adaptor methods for selectively blocking ligation see U.S. Pat. No. 6,197,557 and U.S. Ser. No. 09/910,292 which are incorporated by reference herein in their entirety for all purposes.

Adaptors may also incorporate modified nucleotides that modify the properties of the adaptor sequence. For example, phosphorothioate groups may be incorporated in one of the adaptor strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adaptor strand, or at specified locations within a sequence. A useful option is to sulfurize only the last few residues at each end of the oligo. This results in an oligo that is resistant to exonucleases, but has a natural DNA center.

The term “admixture” refers to the phenomenon of gene flow between populations resulting from migration. Admixture can create linkage disequilibrium (LD).

The term “allele” as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a biopolymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GENECHIP Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” (LD) or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but includes other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but should be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptor is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

A number of methods disclosed herein require the use of one or more “restriction enzymes or endonucleases” to fragment the nucleic acid sample. In general, a restriction enzyme recognizes a specific nucleotide sequence of four to eight nucleotides and cuts the DNA at a site within or a specific distance from the recognition sequence. For example, the restriction enzyme EcoRI recognizes the sequence GAATTC and will cut a DNA molecule between the G and the first A. The length of the recognition sequence is roughly proportional to the frequency of occurrence of the site in the genome. A simplistic theoretical estimate is that a six base pair recognition sequence will occur once in every 4096 (4⁶) base pairs while a four base pair recognition sequence will occur once every 256 (4⁴) base pairs. If an enzyme with a variable position in the recognition site is used this changes the frequency of occurrence. For example, Sty1 has recognition site CCWWGG where W can be A or T so a theoretical estimate for the frequency of occurrence of the site is once every 1024 (4⁴×2²) bases. In silico digestions of sequences from the Human Genome Project show that the actual occurrences may be more or less frequent, depending on the sequence of the restriction site. Because the restriction sites are rare, the appearance of shorter restriction fragments, for example those less than 1000 base pairs, is much less frequent than the appearance of longer fragments. Many different restriction enzymes are known and appropriate restriction enzymes can be selected for a desired result. For a comprehensive list of many commercially available restriction enzymes, their recognition sites and reaction conditions see, New England BioLabs Catalog which is herein incorporated by reference in its entirety for all purposes.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. A “Probe Target Pair” is formed when two macromolecules have combined through molecular recognition to form a complex. c) Methods for genotyping individuals on a genome wide scale

A Single Nucleotide Polymorphism, or SNP, is a small genetic change, or variation, that can occur within a person's DNA sequence. The genetic code is specified by A (adenine), C (cytosine), T (thymine), and G (guanine). SNP variation occurs when a single nucleotide is replaced by one of the other three nucleotides.

On average, SNPs occur in the human population more than 1 percent of the time. Because only about 4 percent of a person's DNA sequence codes for the production of proteins, most SNPs are found outside of “coding sequences”. SNPs found within a coding sequence are of particular interest to researchers because they are more likely to alter the biological function of a protein.

Genetic factors may also confer susceptibility or resistance to a disease and determine the severity or progression of disease. By studying stretches of DNA that have been found to harbor a SNP associated with a disease trait, researchers may begin to reveal relevant genes associated with a disease. Defining and understanding the role of genetic factors in disease will also allow researchers to better evaluate the role non-genetic factors—such as behavior, diet, lifestyle, and physical activity—have on disease.

The sequence listing provides a 33 base sequence for each of two alleles for more than 500,000 human SNPs. Each sequence in SEQ ID NO: 1-1,074,930, is 33 bases in length and includes a SNP base at position 17 plus the 16 bases immediately 5′ of the SNP and the 16 bases immediately 3′ of the SNP. Each allele of the SNP is represented. For example, SEQ ID NO: 1 is identical to SEQ ID NO: 2 except for position 17 which is an A in SEQ ID NO: I and a G in SEQ ID NO: 2:
(SEQ ID NO: 1) GTGACAAGGA TACCAGAAAG TGCACAGGTC TGA
(SEQ ID NO: 2) GTGACAAGGA TACCAGGAAG TGCACAGGTC TGA
   These two sequences represent the two alleles of a biallelic human SNP that can be either an A or a G (T or C on the opposite strand). The sequence of one strand is provided for each. Human genomic DNA is double stranded and one of skill in the art would be able to derive the opposite strand from the sequences provided in the sequence listing. Either strand may be interrogated. The approximately 537,465 SNPs represented by SEQ ID NO: 1-1,074,930 can all be interrogated in a whole genome sampling assay (WGSA) using either Sty I or Nsp I as the restriction enzyme, so each SNP is present on a fragment that is between 200 and 2,000 base pairs when the human genome is digested with Sty I or with Nsp I. The SNPs were selected using information in NCBI build 34. Each SNP can be identified by a SNP ID number, for example, the first SNP represented by SEQ ID NOs: 1 and 2 corresponds to RS ID: 940549.

To interrogate the polymorphism probes are designed to be complementary to one or more regions within the 33 bases provided in SEQ ID NO: 1-1,074,930. In one aspect the probes in a probe set contain a base that corresponds to the polymorphic position and bases up and downstream, for example, in a preferred embodiment a probe set is designed for each SNP and the probe set includes a plurality of 25 base probes that are perfectly complementary to one or the other allele and include the polymorphic position. Probes may be complementary to either strand. A probe set may, for example, perfect match probes that are perfectly complementary to one or the other allele of a SNP and mismatch probes which are perfectly complementary to one or the other allele of a SNP, but for a single mismatch base in the center of the probe. Perfect match probes in a probe set may vary by the position of the probe with reference to the polymorphic position. For example, if the polymorphic position is in the center of the probe the probe is at position 0 in reference to the polymorphism and if the probe is shifted so that the central position of the probe is 4 bases upstream of the SNP the SNP is at position −4. In one aspect the probes in a probe set may be shifted between 1 and 4 bases upstream or downstream of the SNP and may interrogate either strand. There would be 9 different probes possible from each of the 33 base sequences in the sequence listing and 9 possible for the opposite strand, for a total of 36 possible perfect match probes (18×2 alleles). The best performing probes may be selected and a subset of 10-28 perfect match probes may be used, mismatch probes may also be included.

In a particularly preferred embodiment probe sets are designed from at least 800,000 sequences from SEQ ID NOS: 1-1,074,930. In a preferred embodiment the probes of the at least 800,000 probe sets are present on a solid support or a plurality of solid supports so that the location of each probe is known or determinable. The probes of the array include the polymorphic position.

In another aspect a magnesium pyrophosphate precipitate is generated during the extension with TITANIUM Taq Polymerase. The precipitate may interfere with quantification of DNA by optical density analysis and may result in clogging or obstruction of membranes used for purification of nucleic acids. In some aspects it is preferably to treat the PCR amplicon with EDTA to reduce the precipitate prior to taking OD measurements or purification of the DNA. This may be particularly useful when performing the method in an automated fashion, for example, using a robotic system.

In another aspect methods for processing genomic samples in high throughput for hybridization to the disclosed genotyping arrays are disclosed. Methods and workflows for sample preparation in 96 well plates are disclosed.

The disclosed arrays, kits and collections of SNPs may be used, for example, performing association studies to identify genomic regions that are associated with a selected phenotype or phenotypes. In one aspect a set of two arrays to interrogate a set of 500,568 human SNPs is disclosed. The SNPs in the set have an average minor allele frequency (MAF) of 0.21 and an average heterozygosity of 0.29 and about 85% of the human genome is within 10 kb of a SNP in the set, based on analysis in three populations. In one aspect 250 ng of starting genomic DNA is used for each array in the two array set. Whole-genome amplified material prepared by the Qiagen REPLI-G kits may also be used. See U.S. Pat. Nos. 6,617,137 and 6,323,009. Each array in the set includes more than 6.5 million features. Features consist of more than a million copies of a 25 base oligonucleotide probe of known sequence. Each SNP is interrogated by 6 to 10 probe quartets with each probe quartet having a perfect match (PM) and a mistmatch (MM) probe for each allele. In one aspect there are 24 to 40 probes per SNP.

The SNPs were selected for inclusion to optimize coverage of the genome with minimal bias. SNPs were ranked for selection based on LD information and selection was biased toward SNPs with higher MAF. Selection also took into account accuracy, reproducibility and call rate. About 2.2 million SNPs were used for the initial selection, from Caucasian, African and Asian populations (16 each, all from HapMap samples). This resulted in 650,000 SNPs. Using these 650K SNPs 400 individuals were genotyped to maximize for genotyping performance, including call rates, HW and Mendelian error and reproducibility. The final SNPs were selected from this list based on LD and HapMap information and extra SNPs of known genetic importance were included. The median intermarker distance is 2.8 kb, the mean intermarker distance is 5.8 kb. There are more than 225,000 SNPs that are within 10 kb of genes and more than 25,000 SNPs that are in predicted exons or ESTs. There are more than 30,000 human genes that are within 10 kb from at least 1 SNP in the set, that is about 90% of all predicted genes, and more than 18,000 predicted genes are within 10 kb from at least 5 SNPs in the set (about 56% of predicted genes). The set provides more than 80% coverage at r²>0.8 using multi-marker approaches and more than 90% coverage at r²>0.5.

In one aspect the SNPs selected for the whole genome association panel and included in the sequence listing may be supplemented with additional SNPs to increase the genetic power of a genotyping study. For example, the genotype information of the disclosed SNPs may be supplemented in a study with genotype information for a panel of 10,000 to 25,000 or 25,000 to 50,000 tagSNPs or coding SNPs (cSNPs) or a combination of tagSNPs, coding SNPs or genic SNPs. For a discussion of tagSNPs see, for example, Carlson et al., Am J Hum Genet. 2004; 74(1): 106-120. The add on panel of SNPs may be genotyped using any available method. In a preferred aspect the add on panel are genotyped using target specific probes, for example, the MIP assay (see, Hardenbol et al., Genome Res. 15(2):269-75 (2005) and Moorhead et al., Eur. J. Hum Genet 14(2):207-15 (2006). In another aspect an assay based on single base extension (SBE), allele specific primer extension (ASPE), or an oligo ligation based assay (OLA) may be used. For a review of genotyping methods see, for example, Syvanen Nat Rev Genet. (2001) 2(12):930-42.

In another embodiment genotype information obtained using the 500K panel of SNPs disclosed herein may be supplemented with genotype information for a panel of SNPs selected from a genomic region, for example, a chromosome or a region of a chromosome that has been associated with a phenotype.

In another aspect the genotyping arrays include only PM probes, allowing for the same number of SNPs to be interrogated using fewer probes. For example, if a SNP is interrogated with 6 probe quartets, where each quartet includes two PM and two MM probes, if the MM probes are omitted from the array so that the SNP is interrogated by 6 probe pairs (each pair has a PM probe for each allele), the total number of probes for the SNP are 12 instead of 24. The array can thus interrogate the same number of SNPs using about half of the features. In a preferred aspect the array may be a 64 format array. In one aspect the disclosed SNPs are ranked based on performance to identify SNPs that can be reproducibly genotyped using only 12 PM probes. For lower performing SNPs more PM probes may be included, for example, 13 to 24, or 13 to 20 PM probes. In another aspect some SNPs may be interrogated by a mixture of PM and MM probes. For example, some SNPs may be interrogated by about 10, 12, 14, 16, 18 or 20 PM probes and about 2, 4, 6, 8, 10 or 12 MM probes. In some aspects the genotype calls may be made using an algorithm such RLMM which is described in Rabbee and Speed, Bioinformatics 22(1):7-12 (2006), which is incorporated by reference in its entirety. In another aspect an extension of the RLMM algorithm may be used. The BRLMM algorithm has been developed by adding a Bayesian step to RLMM. The Bayesian step provides improved estimates of cluster centers and variances relative to the RLMM algorithm.

The arrays and methods disclosed enable high-powered whole genome association studies, (for example, drug response and disease genetics), cancer studies, linkage studies and population genetics studies. SNPs are the most frequent form of polymorphism in the human genome and it hs been estimated that common SNPs with MAF greater than 10% occur about every 600 bp (Kruglyak and Nickerson, Nat Genet 27:234-236, 2001) resulting in about 5 million common SNPs. Because of the tendency of SNPs that are close together to be correlated because of LD, a subset of all possible common SNPs can be genotyped and that information can be used to infer information about other SNPs that were not genotyped. Statistics for describing LD include D′ and r². See Devlin and Risch Genomics 29:311-322, 1995). If two SNPs are perfectly correlated, meaning there has been no recombination between the two SNPs, then r²=1 and the SNPs are in perfect LD. If two SNPs are in perfect LD, determining the genotype of 1 will give you the genotype of the other. Using an r² threshold of r²>0.8 Carlson et al., were able to use LD-selected tagSNPs to resolve more than 80% of the genome. See Carlson et al., Am J Hum Genet 74:106-20 (2004).

In some aspects the arrays may be used to identify regions of copy number variation, including LOH, amplification and deletion in either the germ line or in tumors. Chromosomal aberrations are frequently identified in tumors. Methods for copy number analysis using genotyping arrays are described, for example, in Huang et al., Bioinformatics, 7:83 (2006), Huang et al., Hum Genomics, 1(4):287-99 (2004) and Bignell et al., Genome Res. 14(2):287-95 (2004). Copy number polymorphisms in the germline may also contribute to genomic variation between normal humans and may be detected using genotyping arrays. See, for example, Sebat et al., Science 305:525-528 (2004). Genotyping arrays provide an advantage to copy number analysis as they can provide allele specific copy number information.

Aspects of the method are further described in the following non-limiting examples.

### EXAMPLES

### Example 1

### GENECHIP 500K-EA Array Protocol

The following reagents and materials were used: reduced EDTA TE Buffer (10 mM Tris HCl, 0.1 mM EDTA, pH 8.0) (TEKNova P/N T0223); Reference Genomic DNA, 103 (50 ng/μl) (Affymetrix P/N 900421); 250 ng Genomic DNA per array at working stock concentration (50 ng/μl); Sty I (10,000 U/ml) obtained from New England Biolab (P/N R0500S) also including NE Buffer 3 from New England Biolab (P/N B7003S to order separately) and BSA from New England Biolab (P/N B9001S to order separately); Nsp I (10,000 U/ml) obtained from New England Biolab (P/N R0602L) including NE Buffer 2 from New England Biolab (P/NB7002S to order separately); Adaptor Nsp 1 (50 μM) Affymetrix P/N 900596 for 30 reactions or 900697 for 100 reactions; Adaptor Sty 1 (50 μM) Affymetrix P/N 900597 for 30 reactions or 900698 for 100 reactions; Molecular Biology Water from BioWhittaker Molecular Applications (Cambrex) (P/N 51200); 96 Well Plate: Bio-Rad (P/N MLP-9601) or Applied Biosystems (P/N 403083); 96-well PLT Clear Adhesive Films obtained from Applied Biosystems (P/N 4306311); 8-Tube Strips, thin wall (0.2 ml) obtained from Bio-Rad (P/N TBS-0201); Strip of 8 Caps obtained from Bio-Rad (P/N TCS-0801); Thermal Cycler; Oligo Control Reagent, 0100 (Affymetrix P/N 900582 or 900581); T4 DNA Ligase obtained from New England Biolab (P/N M0202L) including: T4 DNA Ligase Buffer (P/N B0202S to order separately); Betaine (5M) from Sigma (P/N B0300) or G-C Melt (5M) from Clontech, P/N 639238; dNTP (2.5 mM each) obtained from Takara (P/N 4030), Fisher Scientific, or Invitrogen (P/N R72501); PCR Primer, 002 (100 μM) available at Affymetrix (P/N 900702) (if running Nsp Array), PCR Primer 002 (100 μM) available at Affymetrix (P/N 900595) (if running Sty I Array); Clontech TITANIUM Taq DNA Polymerase (50×) obtained from Clontech (P/N 639209 or 8434-1 containing 50× BD Clontech TITANIUM Taq DNA Polymerase and 10× TITANIUM Taq PCR Buffer; 2% TBE Gel: BMA Reliant precast (2% SEAKEM Gold), Cambrex Bio Science (P/N 54939); All Purpose Hi-Lo DNA Marker: Bionexus Inc. (P/N BN2050) or Direct Load Wide Range DNA Marker from Sigma (P/N D7058); Gel Loading Solution from Sigma (P/N G2526); PCR Tubes (should be comparable and qualified with Bio-Rad DNA ENgine Tetrad, or ABI GeneAmp PCR System). Suitable examples include: individual tubes, Bio-Rad P/N TWI-0201, 8 tube strips, thin-wall (0.2 mL), Bio-Rad P/N TCS-081, 96 well plate, Bio-Rad P/N MLP-9601); and 96 Well PLT Clear Adhesive Films from Applied Biosystems (P/N 4306311).

For PCR purification and elution the following equipment and reagents may be used. Manifold—QIAvac multiwell unit (Qiagen P/N 9014579); EDTA (0.5 M, pH 8.0) Ambion P/N 9260G, DNA Amplification Clean-Up kit, to be used with Affymetrix products, Clontech P/N 636974 (1 plate) or P/N 636975 (4 plates), Biomek Seal and sample aluminum foil lids, Beckman P/N 538619 and a vacuum regulator for use during the PCR clean up step from QIAGEN, P/N 19530. In one embodiment the miniElute 96 UF PCR Purification Kit (Qiagen P/N 28051 or 28053) and Buffer EB (250 ml) (Qiagen P/N 19086); may be used in place of the DNA Amplification Clean-up Kit from Clontech.

The following reagents may be used for the fragmentation and labeling steps: GENECHIP Fragmentation Reagent (DNase I) (Affymetrix P/N 900131); 10× Fragmentation Buffer (Affymetrix P/N 900422 or 900695); 4% TBE Gel: BMA Reliant Precast (4% NuSieve 3:1 Plus Agarose) (Cambrex P/N 54929); GENECHIP DNA Labeling Reagent (30 mM) (Affymetrix P/N 900778 or 900699); Terminal Deoxynucleotidyl Transferase (30 U/μl) (Affymetrix P/N 900508 or 900703); 5× Terminal Deoxynucleotidyl Transferase Buffer (Affymetrix P/N 900425 or 900696); 5M TMACl (Tetramethyl Ammonium Chloride) (Sigma P/N T3411); 10% Tween-20: Pierce (Catalog#: 28320); DMSO (Sigma P/N D5879); MES hydrate SigmaUltra, Sigma (P/N M5287); MES Sodium Salt, Sigma (P/N M5057); 0.5 M EDTA (Ambion, P/N 9260G); 50× Denhardts (Sigma; P/N D2532); HSDNA (Promega P/N D1815); Human Cot-1 (Invitrogen, P/N 15279-011); Oligo Control Reagent, 0100 (OCR, 0100) (Affymetrix P/N 900541); 20×SSPE (Bio Whittaker Molecular Applications, Cambrex, P/N 51214); Acetylated BSA (Invitrogen); SAPE (Streptavidin, R-phycoerythrin conjugate) (Molecular Probes, P/N S866); Biotinylated Anti-Streptavidin (Vector; P/N: BA-0500, 0.5 mg/mL); Distilled Water (Invitrogen P/N 15230147); Acetylated Bovine Serum Albumin (Invitrogen P/N 15561-020); Bleach (5.25% Sodium Hypochlorite) (VWR P/N 21899-504).

Examples of equipment that has been used in the protocol included GENECHIP Fluidics Station 450/250 Affymetrix P/N 00-0079; GENECHIP Hybridization Oven (Affymetrix P/N 800139); GENECHIP Scanner 3000 7G (Affymetrix P/N 00-0205); Affymetrix GENECHIP Operating Software version 1.4 (P/N 690031); Affymetrix GENECHIP Genotyping Analysis Software 4.0 (P/N 690051); GENECHIP Mapping 250K Nsp Array (Affymetrix P/N 520330); and GENECHIP Mapping 250K Sty Array (Affymetrix P/N 520331). PCR thermal cyclers that have been tested include the MJ Research DNA Engine Tetrad PTC-225 with 96 well block (now available from Bio-Rad) and the ABI GENEAMP PCR System 9700 Gold plated 96 well block. The JITTERBUG from Boekel Scientific, model 130000 was also used.

Reagents can also be purchased in a kit form for either the Nsp array (900766 or 900753) or the Sty array (900765 or 900754). The kits each contain the adaptor, the PCR primer, the reference genomic DNA 103, fragmentation reagent, fragmentation buffer, DNA labeling reagent, TdT, and oligo control reagent 0100.

Briefly the method steps used in this example are as follows. A genomic DNA sample concentration is first determined. The sample is then digested into smaller fragments by a restriction enzyme. Then adaptor molecules are then ligated onto the smaller fragments. The ligated fragments are then diluted with water. The diluted fragments are amplified to obtain amplification products. The amplification products are then pooled and purified. The purified amplification product is treated with DNase I for fragmentation, and labeled with Affymetrix DNA Labeling reagent using TdT. Labeled fragments are hybridized overnight (16-18 hours), and then stained with streptavidin/biotinylated anti-streptavidin antibody/SAPE solutions. Then the array is scanned and analyzed for the results.

The first step in the GENECHIP Mapping Assay is determining the genomic DNA concentration for the sample to be genotyped. Then the genomic DNA is diluted to 50 ng/μl using reduced EDTA TE Buffer (0.1 mM EDTA, 10 mM Tris HCl, pH 8.0).

The next step in the process is digestion of the genomic DNA with a restriction enzym. 250 ng genomic DNA is digested with a restriction enzyme. Examples of restriction enzymes used include Nsp I, Sty I, Eco RI, Xba I, Bgl II, Bsr GI, Basj I, and Tsp45. In our example, Nsp I or Sty I restriction enzymes are used in the assay. For the digestion procedure, the following digestion master mix is prepared on ice depending on the restriction enzyme being used. For multiple samples, about 5% excess may be prepared. For the Nsp I restriction enzyme, the digestion master mix is prepared on ice by mixing 9.75 μl of water, 2 μl of NE buffer 2 (10×), 2 μl of BSA (10×(1 mg/ml)), and 1 μl Nsp I (10 U/μl). For the Sty I restriction enzyme, the digestion master mix is prepared on ice by mixing 9.75 μl of water, 2 μl of NE buffer 3 (10×), 2 μl BSA (10×(1 mg/ml)), and 2 μl Sty I (10U/μL). After the digestion master mix for either restriction enzyme is made, 15 μl of the master mix is added to 5 μl of genomic DNA (50 ng/μl).

The next step in the process is the ligation step. The smaller fragments of genomic DNA are ligated to adaptor sequences. For the ligation procedure, a ligation master mix on ice is prepared. For multiple samples, prepare a 5% excess. For the Nsp I restriction enzyme, the Ligation Master Mix on ice consists of 0.5 μl of Adaptor Nsp I (50 μM), 2.5 μl of T4 DNA Ligase buffer (10×) kept on ice, 2 μl of T4 DNA Ligase (400 U/μL) and mixed well. For the Sty I restriction enzyme, the Ligation Master Mix on ice consists of 0.5 μl of Adaptor Sty I (50 μM), 2.5 μl of T4 DNA Ligase buffer (10×) kept on ice, 2 μl of T4 DNA Ligase (400 U/μl) and mixed well. After the Ligation Master Mix for either restriction enzyme is made, add 5 μl of the Ligation Master Mix to 20 μl of digested DNA.

The next step in the process is the dilution step. The ligated DNA is then diluted with water. For the dilution step, dilute 25 μl of the ligated DNA with 75 μl of water.

The next step in the process is the amplification step. For the PCR procedure, prepare a PCR Master Mix on Ice (3 PCR reactions per sample) for Nsp I or Sty I ligation reactions. For multiple samples, prepare a 5% excess. The PCR Master Mix On Ice for 1 PCR reaction consists of 46 μl of water, 10 μl of BD Titanium Taq PCR Buffer (10×), 24 μl of Betaine (5M), 10 μl dNTP (2.5 mM each), 4 μl of PCR Primer (100 μM), and 1 μl Titanium Taq DNA Polymerase (50X). Then add 5 μl of diluted ligated DNA from the ligation step to 95 μl of the PCR Master Mix. Three PCR reactions are needed to produce sufficient product for hybridization to one array (each reaction=100 μl). Then the amplification products are purified to obtain a Purified PCR product.

The next step in the process is the fragmentation step. For the fragmentation step, add 5 μL of 10× Fragmentation Buffer to 45 μl of Purified PCR product (60 μg in EB Buffer) on the fragmentation plate on ice.

The next step in the process is the dilution step. The fragmentation reagent should be diluted to 0.04 U/μl. There are two examples of dilution listed for two different concentrations of Fragmentation reagent. The first example is for 2 U/μl and this dilution consists of 3 μl of Fragmentation Reagent, 15 μl of 10× Fragmentation Buffer, and 132 μl of water mixed well. The second example is for 3 U/μl and this dilution consists of 2 μl of Fragmentation Reagent, 15 μl of 10× Fragmentation Buffer, and 133 μl of water mixed well. Finally, the Fragmentation Mix needs to be mixed with the diluted Fragmentation Reagent. This fragmentation dilution consists of 50 μl of Fragmentation Mix and 5 μl of Diluted Fragmentation Reagent (0.04 U/μl).

The next step in the process is the labeling step. For the Labeling step, prepare a Labeling Mix as Master Mix on ice. For multiple samples, prepare a 5% excess. The Labeling Mix consists of 14 μl of 5× TdT Buffer, 2 μl GENECHIP DNA Labeling Reagent (15 mM), and 3.5 μl of TdT (30 U/μL) and mixed well. Then 19.5 μl of the Labeling Mix is added to 50.5 μl fragmented DNA from the fragmentation step.

The next step in the process is the hybridization step. For the hybridization step, prepare a Hybridization Cocktail Master Mix. For multiple samples, prepare a 5% excess. Before the Hybridization Master can be prepared, a 12× MES Stock Buffer needs to be prepared first. The 12× MES Stock Buffer (1.25 M MES, 0.89M [NA+]) is prepared by mixing 70.4 g MES hydrate, 193.3. MES Sodium Salt, 800 ml Molecular Biology Grade Water and adjusting volume to 1,000 ml. The pH should be 6.5-6.7 and the mixture is filtered through a 0.2 μm filter. The Hybridization Cocktail Master Mix consists of 12 μl of MES (12×; 1.22M), 13 μl DMSO (100%), 13 μl of Denhardt's Solution (50×), 3 μl of EDTA (0.5M), 3 μl of HSDNA (10 mg/ml), 2 μl of OCR (0100), 3 μl Human Cot-1 (1 mg/ml), 1 μl Tween-20 (3%), and 140 μl TMACl (5M) and mixed well. Then 190 μl of the Hybridization Mix is added to 70 μl of the labeled DNA.

The next step in the process is the washing and staining steps. Before these steps can be performed, two wash buffers, an Anti-Streptavidin antibody, a stock buffer, array buffers, a stain buffer, SAPE solution, and an antibody solution need to be prepared first. For Wash Buffer A (Non-Stringent Buffer Wash Buffer) (6×SSPE, 0.01% Tween 20), prepare 300 ml of 20×SSPE, 1.0 ml of 10% Tween-20, and 699 ml of water. Filter Wash A Buffer through a 0.2 μm filter and store at room temperature. For Wash Buffer B (Stringent Wash Buffer) (0.6×SSPE, 0.01% Tween-20), prepare 30 ml of 20×SSPE, 1.0 ml of 10% Tween-20, and 969 ml of water. Filter Wash B Buffer through a 0.2 μm filter and store at room temperature. For the Anti-Streptavidin Antibody, resuspend 0.5 mg in 1 ml of water and store at 4° C. For the 12×MES Stock Buffer (1.25M MES, 0.89M [Na+]), prepare 70.4 g of MES hydrate, 193.3 g of MES Sodium Salt, 800 ml of Molecular Biology Grade Water, mix well and adjust volume to 1,000 ml. The pH should be between 6.5-6.7. Filter mixture through a 0.2 μm filter. For the 1× Array Holding Buffer (Final 1× concentration is 100 mM MES, 1M [NA+], 0.01% Tween-20), prepare 8.3 mL of 12× MES Stock Buffer, 18.5 ml of 5M NaCl, 0.1 ml of 10% Tween-20, 73.1 ml of water. Store at 2° C.-8° C. and shield the mixture from light. The Stain Buffer consists of 666.7 μl of water, 300 μl SSPE (20×), 3.3 Tween-20 (3%), and 20 μl Denhardt's (50×) and then split this mixture by one-half (½). To prepare the SAPE Stain Solution, add 5 μl of 1 mg/mL Streptavidin Phycoerythrin (SAPE) with 495 μl of Stain Buffer. To prepare the Antibody Stain Solution, add 5 μl 0.5 mg/ml biotinylated antibody to 495 μl of Stain Buffer. To prepare the Array Holding Buffer, add 8.3 ml of MES Stock Buffer (12×) Buffer, 18.5 ml of 5M NaCl, 0.1 mL of Tween-20 (10%), and 73.1 ml of water.

After the solutions are prepared, the washing and staining steps occur in the following order. First, a post hybridization wash is performed for 6 cycles of 5 mixes/cycle with Wash Buffer A at 25° C. Then a second post hybridization wash is performed for 24 cycles of 5 mixes/cycle with Wash Buffer B at 45° C. The probe array is stained for 10 minutes in SAPE solution at 25° C. Then the array is washed with 6 cycles of 5 mixes/cycle with Wash Buffer A at 25° C. The array is stained for 10 minutes in antibody solution at 25° C. The probe array is then stained for 10 minutes in SAPE solution at 25° C. Then the array goes through a final wash for 10 cycles of 6 mixes/cycle with Wash Buffer A at 30° C. Then the array is filled with Array Holding Buffer.

The final step in the process is the detection step. The array is scanned and analyzed onto a GENECHIP array using GENECHIP Operating Software (GCOS) and GENECHIP DNA Analysis Software (GDAS).

There are many variables in the GENECHIP 500K-EA Assay protocol which can affect the efficacy of the process. Some of these variables include adaptor concentration, amount of DNA, PCR Primer concentration and PCR extension time. Additionally, some of the variables of the hybridization process which can affect the process include the percent of DMSO in the cocktail master mix, the percent of Denhardt's Solution in the cocktail master mix, the percent of Human Cot-1 in the cocktail master mix, and the temperature. Some of the variables of the stringent washing process include the numbers of cycles, the temperature, and the salt concentration.

Some of the SNP selection factors include the Call Rate, minor allele frequency (MAF), pHW, Nsp I or StyI Fragment Type, and Proximity to Nearby SNPs. The GENECHIP 500K-EA Set has been optimized to accurately detect greater than 500,000 SNPs in each sample. The assay reduces the complexity of the genome by preferentially amplifying approximately 200-1100 bp Nsp I fragments and approximately 200-1100 bp Sty I fragments using a single PCR primer from only 250 ng DNA per restriction enzyme. Two assays using the protocol above were performed. The first assay performed for the detection greater than 500,000 SNPs set featured a 5 micron feature size, 550 mB assay complexity, 24 probes/SNP, 1.09 pixel size, and used a prototype grid software. The second assay performed for the detection greater than 500,000 SNPs set featured a 5 micron feature size, 550 megabase assay complexity, 56 probes/SNP, 0.7 pixel size, and GCOS 1.3 for alignment size. The second assay also illustrated that Titanium Polymerase may function better than the Taq Gold Polymerase in the amplification step. For the second test, the following changes were introduced: hybridization temperature was increased from 48° C. to 49° C., stringent wash was increased from 6 to 24 cycles, and labeling was reduced from 4 to 2 hours.

Oligonucleotide sequences that are preferably used in the assay include:

| | |
|---|---|
| 5′ ATTATGAGCACGACAGACGCCTGATC | (SEQ ID NO: 1074931) |
| T 3′ | |
| (PCR PRIMER 002 and Adaptor | |
| Sty I top PN 100485); | |
| 5′-[Phos] AGATCAGGCGTCTGTCGTG | (SEQ ID NO: 1074932) |
| CTCATAA-3′ | |
| PN 100520 Adaptor Nsp I | |
| bottom; | |
| 5′-[Phos] CWWGAGATCAGGCGTCTGT | (SEQ ID NO: 1074933) |
| CGTGCTCATAA-3′ | |
| PN 100521 Adaptor Sty I | |
| bottom | |
| and | |
| 5′-ATTATGAGCACGACAGACGCCTGATC | (SEQ ID NO: 1074934) |
| TCATG-3′, | |
| Nsp I top PN 100522 | |

SEQ ID NOS: 1074932 and 1074934 are complementary over the length of SEQ ID NO: 1074932 and can hybridize to form a double stranded adaptor with a 3′ overhang (3′-GTAC-5′) that is complementary to the overhang left by Nsp I digestion. This Nsp I adaptor with sticky end can be efficiently ligated to the ends of fragments digested with Nsp I. In a preferred aspect the 5′ end of SEQ ID NO: 1074932 is phosophorylated to facilitate ligation.

SEQ ID NOS: 1074931 and 1074933 are complementary and can be hybridized to form an adaptor that is partially double stranded and has a single stranded 5′ overhang that is complementary to the 5′ overhang resulting from digestion with Sty I. The overhang is preferably phosophorylated at the 5′ end to facilitate ligation. SEQ ID NO: 1074933 includes two positions that are partially degenerate, represented by “W”, and can be either A or T. The oligo is a mixture of different sequences that have one of the following combinations at the WW position: AT, AA, TT, or TA.

### Example 2

### Human Mapping 500K 96-Well Plate Protocol

The protocol described below involves enzymatic reactions that are optimized for the reaction conditions provided, thus it is important to control and monitor variables such as pH, salt concentration, time and temperature. For additional details of the protocol please see the GENECHIP Mapping 500K Assay Manual (P/N 701930 Rev. 3) which is incorporated herein by reference in its entirety.

Stage 1-Genomic DNA Plate Preparation. This protocol has been optimized using UV absorbance to determine genomic DNA concentrations. Other quantitation methods such as PicoGreen will give different readings. Therefore, you should convert readings from other methods to the equivalent UV absorbance reading. To prepare the genomic DNA plate: Thoroughly mix the genomic DNA by vortexing at high speed for 3 sec. Determine the concentration of each genomic DNA sample. Based on OD measurements, dilute each sample to 50 ng/μL using reduced EDTA TE buffer (10 mM Tris HCL, 0.1 mM EDTA, pH 8.0). Apply the convention that 1 absorbance unit at 260 nm equals 50 μg/mL for double-stranded DNA. This convention assumes a path length of 1 cm. Consult your spectrophotometer handbook for more information. If using a quantitation method other than UV absorbance, convert the reading to the equivalent UV absorbance reading. Thoroughly mix the diluted DNA by vortexing at high speed for 3 sec.

To aliquot the prepared genomic DNA: Vortex the plate of genomic DNA at high speed for 10 sec, then spin down at 2000 rpm for 30 sec. Aliquot 5 μL of each DNA to the corresponding wells of a 96-well reaction plate. 5 μL of the 50 ng/μL working stock is equivalent to 250 ng genomic DNA per well. For this protocol, one plate is required to process Nsp samples; a second plate is required to process Sty samples. For best results, do not process Nsp and Sty samples on the same day. If continuing immediately to the next stage, place the plate with prepared genomic DNA in a double cooling chamber on ice. Otherwise, seal each plate with adhesive film. Do one of the following: Proceed to the next stage, processing one plate of samples, one enzyme at a time. Store the sealed plates of diluted genomic DNA at −20° C.

Stage 2-Restriction Enzyme Digestion During this stage, the genomic DNA is digested by one of two restriction enzymes: Nsp I or Sty I. You will prepare the Digestion Master Mix, then add it to the samples. The samples are then placed onto a thermal cycler and the 500K Digest program is run. The input required from Stage 1: Genomic DNA Plate Preparation is: 1 Plate, 96-well Genomic DNA prepared as instructed in the previous stage (5 μL at 50 ng/uL in each well). Keep in a cooling chamber on ice.

Allow the following reagents to thaw on ice: NE Buffer and BSA. If the plate of genomic DNA from stage 1 was frozen, allow it to thaw in a cooling chamber on ice. To prepare the work area: Place a double cooling chamber and a cooler on ice. Label the following tubes, then place in the cooling chamber: One strip of 12 tubes labeled Dig A 2.0 mL Eppendorf tube labeled Dig MM Place the ACCUGENE water on ice. Place the plate of prepared genomic DNA from Stage 1 in the cooling chamber. To prepare the reagents (except for the enzyme): Vortex 3 times, 1 sec each time. Pulse spin for 3 sec. Place in the cooling chamber. Power on the thermal cycler to preheat the lid. Leave the block at room temperature. For best results, the same team or individual operator should not process samples with both Nsp and Sty enzymes on the same day. Best practice is to process samples for either Nsp or Sty on a given day. Keeping all reagents and tubes on ice, prepare the Digestion Master Mix as follows: To the 2.0 mL Eppendorf tube, add the appropriate volumes of the following reagents based on the enzyme you are using, ACCUGENE Water, NE Buffer, and BSA. Remove the appropriate enzyme (Nsp I or Sty I) from the freezer and immediately place in a cooler. Pulse spin the enzyme for 3 sec. Immediately add the enzyme to the master mix, then place remaining enzyme back in the cooler. Vortex the master mix at high speed 3 times, 1 sec each time. Pulse spin for 3 sec. Place in the cooling chamber. Return any remaining enzyme to the freezer. Proceed immediately to Add Digestion Master Mix to Samples. The master mix for each enzyme is given per reaction and for a mix for 96 samples with an extra 15%. For Nsp I Digestion Master Mix use ACCUGENE Water 11.55 μL for 1 sample or 1275.1 μL for 96 samples, NE Buffer 2 (10×) 2 μL for 1 sample or 220.8 μL for 96 samples, BSA (100×; 10 mg/mL) 0.2 μL for 1 sample or 22.1 μL for 96 samples, Nsp I (10 U/μL) 1 μL per sample or 110.4 μL for 96 samples for a Total per reaction of 14.75 μL and total for 96 samles of 1628.4 μL. For Sty I Digestion Master Mix Reagent: ACCUGENE Water 11.55 μL per reaction, 1275.1 μL for 96 reaction mix, NE Buffer 3 (10×) 2 μL per reaction, 220.8 μL for 96 reaction mix, BSA (100×; 10 mg/mL) 0.2 μL per reaction or 22.1 μL for 96 reaction mix, Sty I (10 U/μL) 1 μL per reaction or 110.4 μL for 96 reaction mix. The Total per reaction is 14.75 μL and the total for the 96 reaction mix is 1628.4 μL.

To add Digestion Master Mix to samples: Using a single channel P200 pipette, aliquot 135 μL of Digestion Master Mix to each tube of the strip tubes labeled Dig. Using a 12-channel P20 pipette, add 14.75 μL of Digestion Master Mix to each DNA sample in the cooling chamber on ice. The total volume in each well is now 19.75 μL. Genomic DNA (50 ng/μL) 5 μL Digestion Master Mix 14.75 μL Total Volume 19.75 μL. Seal the plate tightly with adhesive film. Vortex the center of the plate at high speed for 3 sec. Spin down the plate at 2000 rpm for 30 sec. Ensure that the lid of thermal cycler is preheated. Load the plate onto the thermal cycler and run the 500K Digest program. 37° C. 120 minutes 65° C. 20 minutes 4° C. Hold When the program is finished, remove the plate and spin it down at 2000 rpm for 30 sec. Do one of the following: If proceeding directly to the next step, place the plate in a cooling chamber on ice. If not proceeding directly to the next step, store the samples at −20° C.

Stage 3-Ligation. During this stage, the digested samples are ligated using either the Nsp or Sty Adaptor. Prepare the Ligation Master Mix, then add it to the samples. The samples are then placed onto a thermal cycler and the 500K Ligate program is run. When the program is finished, dilute the ligated samples with ACCUGENE water. The input required from Stage 2: Restriction Enzyme Digestion is: 1 Plate of digested samples in a cooling chamber on ice. 1 vial T4 DNA Ligase (400 U/μL; NEB) 1 vial T4 DNA Ligase Buffer (10×) 1 vial Adaptor, Nsp or Sty as appropriate (50 μM) 10 mL ACCUGENE water, molecular biology-grade Aliquot the T4 DNA Ligase Buffer (10×) after thawing for the first time to avoid multiple freeze-thaw cycles. See vendor instructions. Be sure to use the correct adaptor (Nsp or Sty). To thaw the reagents and Digestion Stage Plate: Allow the following reagents to thaw on ice: Adaptor Nsp I or Sty I as appropriate T4 DNA Ligase Buffer (10×) Takes approximately 20 minutes to thaw. If the Digestion Stage plate was frozen, allow to thaw in a cooling chamber on ice. To prepare the work area: Place a double cooling chamber and a cooler on ice. Label the following tubes, then place in the cooling chamber: One strip of 12 tubes labeled Lig, a 2.0 mL Eppendorf tube labeled Lig MM and a solution basin Prepare the Digestion Stage plate as follows: A. Vortex the center of the plate at high speed for 3 sec. B. Spin down the plate at 2000 rpm for 30 sec. C. Place back in the cooling chamber on ice. To prepare the reagents: Vortex at high speed 3 times, 1 sec each time (except for the enzyme). Pulse spin for 3 sec. Place in the cooling chamber. T4 DNA Ligase Buffer (10×) contains ATP and should be thawed on ice. Vortex the buffer as long as necessary before use to ensure precipitate is re-suspended and that the buffer is clear. Avoid multiple freeze-thaw cycles per vendor instructions. Power on the thermal cycler to preheat the lid. Leave the block at room temperature. The lid should be preheated before samples are loaded. Keeping all reagents and tubes on ice, prepare the Ligation Master Mix as follows: To the 2.0 mL Eppendorf tube, add the following reagents based on the volumes shown below depending on the enzyme: Adaptor (Nsp or Sty) and T4 DNA Ligase Buffer (10×) Remove the T4 DNA Ligase from the freezer and immediately place in the cooler on ice. Pulse spin the T4 DNA Ligase for 3 sec. Immediately add the T4 DNA Ligase to the master mix; then place back in the cooler. Vortex the master mix at high speed 3 times, 1 sec each time. Pulse spin for 3 sec. Place the master mix on ice. Proceed immediately to Add Ligation Master Mix to Reactions. Nsp I Ligation Master Mix given for 1 Sample or for 96 Sample mix (15% extra). Adaptor Nsp 1 (50 μM) 0.75 μL or 82.8 μL, T4 DNA Ligase Buffer (10×) 2.5 μL or 276 μL, T4 DNA Ligase (400 U/μL) 2 μL or 220.8 μL. The total is 5.25 μL or 579.6 μL. For Sty I Ligation Master Mix mix Adaptor Sty 1 (50 μM) 0.75 μL or 82.8 μL, T4 Ligase Buffer (10×) 2.5 μL or 276 μL, T4 DNA Ligase (400U/μL) 2 μL or 220.8 μL for a Total 5.25 μL 579.6 μL To add Ligation Master Mix to samples: Using a single channel P100 pipette, aliquot 48 μL of Ligation Master Mix to each tube of the strip tubes on ice. Using a 12-channel P20 pipette, aliquot 5.25 μL of Ligation Master Mix to each reaction on the Digestion Stage Plate. Digested DNA 19.75 μL Ligation Master Mix* 5.25 μL Contains ATP and DTT. Keep on ice. Total 25 μL. Seal the plate tightly with adhesive film. Vortex the center of the plate at high speed for 3 sec. Spin down the plate at 2000 rpm for 30 sec. Ensure that the thermal cycler lid is preheated. Load the plate onto the thermal cycler and run the 500K Ligate program. 16° C. 180 minutes 70° C. 20 minutes 4° C. Hold To dilute the samples: Place the ACCUGENE Water on ice 20 minutes prior to use. When the 500K Ligate program is finished, remove the plate and spin it down at 2000 rpm for 30 sec. Place the plate in a cooling chamber on ice. Dilute each reaction as follows: Pour 10 mL ACCUGENE water into the solution basin. Using a 12-channel P200 pipette, add 75 μL of the water to each reaction. The total volume in each well is 100 μL. Ligated DNA 25 μL ACCUGENE water 75 μL Total 100 μL Seal the plate tightly with adhesive film. Vortex the center of the plate at high speed for 3 sec. Spin down the plate at 2000 rpm for 30 sec. Do one of the following: If proceeding to the next step, store the plate in a cooling chamber on ice for up to 60 minutes. If not proceeding directly to the next step, store the plate at −20° C.

Stage 4: During this stage, equal amounts of each ligated sample are transferred into three new 96-well plates. Then prepare the PCR Master Mix, and add it to each sample. Each plate is placed onto a thermal cycler and the 500K PCR program is run. When the program is finished, check the results of this stage by running 3 μL of each PCR product on a 2% TBE gel. Samples can be held overnight. The input required from Stage 3: Ligation is: 1 Plate of diluted ligated samples in a cooling chamber on ice. Equipment and Consumables Required for Stage 4: PCR: 1 Cooler, chilled to −20° C. 2 double or 4 single Cooling chambers, chilled to 4° C. (do not freeze) 1 Ice bucket, filled with ice 1 Marker, fine point, permanent 1 Microcentrifuge 1 Pipette, single channel P20 1 Pipette, single channel P100 1 Pipette, single channel P200 1 Pipette, single channel P1000 1 Pipette, 12-channel P20 1 Pipette, 12-channel P200 As needed Pipette tips for pipettes listed above; full racks 6 Plates, 96-well reaction** 1 Plate centrifuge 7 Plate seal, 1 Solution basin, 55 mL 3 Thermal cycler, 1 Falcon 50 mL tube, 1 Vortexer

The following reagents are required for this stage. The amounts listed are sufficient to process one full 96-well reaction plate. Reagents Required for Stage 4: PCR: 15 mL ACCUGENE water, molecular biology-grade 875 μL (2 vials) PCR Primer 002 (100 μM). The following reagents from the TITANIUM™ DNA Amplification Kit: 1.28 mL (4 vials) dNTPs (2.5 mM each), 1 mL (7 vials) GC-Melt (5M), 100 μL (7 vials) TITANIUM Taq DNA Polymerase (50×), and 600 μL (6 vials) TITANIUM Taq PCR Buffer (10×)

The following gels and related materials are required for this stage. The amounts listed are sufficient to process one full 96-well reaction plate. To help ensure the best results, carefully read the information below before beginning this stage of the protocol. Make sure the ligated DNA was diluted to 100 μL with ACCUGENE water. Prepare PCR Master Mix immediately prior to use, and prepare in Pre-PCR Clean room. To help ensure the correct distribution of fragments, be sure to add the correct amount of primer to the master mix. Mix the master mix well to ensure the even distribution of primers. Set up the PCRs in PCR Staging Area. To ensure consistent results, take 3 μL aliquots from each PCR to run on gels before adding EDTA. Gels and Related Materials Required for Stage 4: PCR: 50 μL DNA Marker 5 Gels, 2% TBE As needed Gel loading solution and 3 96-well reaction plates. A PCR negative control can be included in the experiment to assess the presence of contamination.

Allow the following reagents to thaw on ice. TITANIUM Taq PCR Buffer dNTPs, PCR Primer 002 If the Ligation Stage plate was frozen, allow to thaw in a cooling chamber on ice. To prepare the work area: place two double or four single cooling chambers and one cooler on ice. Label the following, then place in a cooling chamber: Three 96-well reaction plates labeled P1, P2, P3. A 50 mL Falcon tube labeled PCR MM. Place on ice: ACCUGENE water, GC-Melt, and a solution basin. Leave the TITANIUM Taq DNA Polymerase at −20° C. until ready to use. Prepare the Ligation Stage plate as follows: Vortex the center of the plate at high speed for 3 sec. Spin down the plate at 2000 rpm for 30 sec. Place back in the cooling chamber on ice. Label the plate Lig. To prepare the reagents: Vortex at high speed 3 times, 1 sec each time (except for the enzyme). Pulse spin for 3 sec. Place in a cooling chamber. Preheat the Thermal Cycler Lids (Main Lab). The lids should be preheated before loading samples; leave the blocks at room temperature. If preparing the plates for PCR, it is best not to go from the Pre-PCR Room or Staging Area to the Main Lab and then back again. To add DNA to the reaction plates: Working one row at a time and using a 12-channel P20 pipette, transfer 10 μL of sample from each well of the Ligation Plate to the corresponding well of each reaction plate. Transfer 10 μL of sample from each well of row A on the Ligation Plate to the corresponding wells of row A on reaction plates P1, P2 and P3. Seal each plate with adhesive film, and leave in cooling chambers on ice.

Transferring Equal Aliquots of Diluted, Ligated Samples to Three Reaction Plates P1 P2 P3 B Ligation Stage Plate An equal aliquot of each sample from the Ligation Stage Plate is transferred to the corresponding well of each PCR Plate. For example, an equal aliquot of each sample from row A on the Ligation Stage Plate is transferred to the corresponding wells of row A on PCR Plates P1, P2 and P3. Reaction Plate P1 Reaction Plate P2 Reaction Plate P3.

Prepare enough PCR Master Mix to run three PCR reactions per sample. Location Pre-PCR Clean Room Prepare the PCR Master Mix To prepare the PCR Master Mix: Keeping the 50 mL Falcon tube in the cooling chamber, add the reagents in the order shown. Remove the TITANIUM Taq DNA Polymerase from the freezer and immediately place in a cooler. Pulse spin the Taq DNA polymerase for 3 sec. Immediately add the Taq DNA polymerase to the master mix; then return the tube to the cooler on ice. Vortex the master mix at high speed 3 times, 1 sec each time. Pour the mix into the solution basin, keeping the basin on ice. The PCR reaction is sensitive to the concentration of primer used. It is critical that the correct amount of primer be added to the PCR Master Mix to achieve the correct distribution of fragments (200 to 1100 bp) in the products. Check the PCR reactions on a gel to ensure that the distribution is correct (see FIG. 4.3). 90 μg of PCR product is needed for fragmentation.

To add PCR Master Mix to samples: Using a 12-channel P200 pipette, add 90 μL PCR Master Mix to each sample. The total volume in each well is 100 μL. Seal each reaction plate tightly with adhesive film. Vortex the center of each reaction plate at high speed for 3 sec. Spin down the plates at 2000 rpm for 30 sec. Keep the reaction plates in cooling chambers on ice until loaded onto the thermal cyclers. Master Mix Reagent For 1 Reaction or For 3 PCR Plates (15% extra) ACCUGENE water 39.5 μL 13.082 mL TITANIUM Taq PCR Buffer (10×) 10 μL 3.312 mL GC-Melt (5M) 20 μL 6.624 mL dNTP (2.5 mM each) 14 μL 4.637 mL PCR Primer 002 (100 μM) 4.5 μL 1.490 mL TITANIUM Taq DNA Polymerase (50×) 2 μL 0.663 mL Total 90 μL 29.808 mL

To load the plates and run the 500K PCR program: transfer the reaction plates to the Main Lab. Ensure that the thermal cycler lids are preheated. The block should be at room temperature. Load each reaction plate onto a thermal cycler. Run the 500K PCR program. The program varies depending upon the thermal cyclers being used. PCR protocols for the MJ Tetrad PTC-225 and Applied Biosystems thermal cyclers are different. If using GENEAMP PCR System 9700 thermal cyclers, be sure the blocks are silver or gold-plated silver. For best results, do not use thermal cyclers with aluminum blocks. It is not easy to visually distinguish between silver and aluminum blocks.

500K PCR Thermal Cycler Program for the GENEAMP PCR System 9700 (silver or gold-plated silver blocks) 500K PCR Program for a GENEAMP PCR System 970: 94° C. for 3 minutes; 94° C. for 30 sec, 60° C. for 45 sec, and 68° C. for 15 sec for 30 cycles, then 68° C. for 7 minutes and 4° C. HOLD (can be held overnight) Volume: 100 μL Specify Maximum mode. 500K PCR Program for MJ Tetrad PTC-225 94° C. for 3 minutes; 94° C. for 30 sec, 60° C. for 30 sec, and 68° C. for 15 sec for 30 cycles, then 68° C. for 7 minutes, then 4° C. HOLD (can be held overnight) Volume: 100 μL Use Heated Lid and Calculated Temperature.

To ensure consistent results, take 3 μL aliquot from each PCR before adding EDTA. Run the Gels When the 500K PCR program is finished: Remove each plate from the thermal cycler. Spin down plates at 2000 rpm for 30 sec. Place plates in cooling chambers on ice or keep at 4° C. Label three fresh 96-well reaction plates P1Gel, P2Gel and P3Gel. Aliquot 3 μL of 2× Gel Loading Dye to each well of the three plates. Using a 12-channel P20 pipette, transfer 3 μL of each PCR product from plates P1, P2 and P3 to the corresponding plate, row and wells of plates P1Gel, P2Gel and P3Gel. Example: 3 μL of each PCR product from each well of row A on plate P1 is transferred to the corresponding wells of row A on plate P1Gel. Seal plates P1Gel, P2Gel and P3Gel. Vortex the center of plates P1Gel, P2Gel and P3Gel, then spin down at 2000 rpm for 30 sec. Load all 6 μL from each well of plates P1Gel, P2Gel and P3Gel onto 2% TBE gels. Run the gels at 120V for 40 minutes to 1 hour. Verify that the PCR product distribution is between ˜250 bp to 1100 bp. 90 μg of PCR product is needed for fragmentation. Wear the appropriate personal protective equipment when handling ethidium bromide. Proceed to the next stage within 60 minutes or seal the plates with PCR product and store at −20° C. Average product distribution is between ˜250 to 1100 bp.

Stage 5: PCR Product Purification and Elution: The input required from Stage 4: PCR is: 3 Plates of PCR product in cooling chambers on ice. The following equipment and consumables are required for this stage: PCR Product Purification and Elution, 1 JITTERBUG, Kimwipes, 1 Manifold, QIAvac Multiwell, 1 Marker, fine point, permanent, 1 Pipette, single channel P200, 1 Pipette, single channel P1000, 1 Pipette, 12-channel P20, 1 Pipette, 12-channel P200, Pipette tips for pipettes listed above; full racks, 1 Plate, 96-well PCR, 1 Plate centrifuge, 1 Plate, Clontech Clean-Up 4 Plate holders, 5 Plate seal, 4 Plate supports, 1 Regulator (QIAGEN), 1 Solution basin, 55 mL and 1 Vortexer.

The following reagents are used for this stage. The amounts listed are sufficient to process one full 96-well reaction plate. For best results, carefully read the information below before beginning this stage of the protocol. The working stock of EDTA should be diluted to 0.1 M before use. For best results the ACCUGENE water should be used for this stage. Using in-house ddH2O is can negatively impact downstream stages, particularly Stage 7: Fragmentation. The fragmentation reaction is very sensitive to pH and metal ion contamination. To avoid cross-contamination and the introduction of air bubbles, pipette very careful when pooling the three PCR reactions for each sample onto the Clontech Clean-Up Plate. Maintain the vacuum at 600 mbar. Reagents Required for Stage 5: PCR Product Purification and Elution: 1 Clean-Up Plate (Clontech) 3 mL EDTA, diluted to 0.1M (working stock is 0.5 M, pH 8.0) 5 mL RB Buffer 75 mL ACCUGENE water, molecular biology-grade. The PCR reactions contain significant contaminants including EDTA. These contaminants can affect subsequent steps unless removed by washing. Therefore, be sure to perform three water washes. After the third wash, the wells should be completely dry before eluting the samples with RB Buffer. Any extra water carried with the RB Buffer to the next stage can result in over-fragmentation. Immediately upon removal from the manifold, blot the bottom of the plate and wipe the bottom of each well. Any remaining liquid will quickly seep back into the wells.

To prepare the PCR Product Plates from the previous stage: Place the three PCR product plates on the bench top in plate holders. If frozen, allow them to thaw to room temperature. Once at room temperature, vortex the center of each plate at high speed for 3 sec. Spin down each plate at 2000 rpm for 30 sec. Dilute the Working Solution of EDTA Dilute the working stock of EDTA to a concentration of 0.1 M. A higher concentration may interfere with downstream steps. To set up the manifold: Connect the manifold and regulator to a suitable vacuum source able to maintain 600 mbar. Place the waste tray inside the base of the manifold. Do not turn on the vacuum at this time.

To add diluted EDTA to the PCR products: Add 3 mL of diluted EDTA (0.1M) to a solution basin. Using a 12-channel P20 pipette, aliquot 8 μL of diluted EDTA to each well with PCR product on each PCR product plate. Tightly seal each plate and vortex the center of each plate at high speed for 3 sec. Spin down each plate at 2000 rpm for 30 sec. Place each plate back in a plate holder. PREPARE THE CLEAN-UP PLATE Follow the steps as described below. Consult the Clontech Clean-Up Plate Handbook for the general procedure. To prepare the Clean-Up Plate: Label the plate to indicate its orientation CUP BL (Clean-Up Plate bottom left). If not processing a full plate of samples, cover the wells that will not be used with adhesive film as follows: Apply pressure around the edges of the plate to make the film stick. B. Cut the film between the used and unused wells. Remove the portion that covers the wells to be used.

Working one row at a time, pool the PCR products as follows: Cut the adhesive film from the first row of each reaction plate. Using a 12-channel P200 pipette, transfer and pool the samples from the same row and well of each PCR product plate to the corresponding row and well of the Clean-Up Plate. Transfer each sample from row A of plates P1, P2 and P3 to the corresponding wells of row A on the Clean-Up Plate. To avoid piercing the membrane, do not pipette up and down in the Clean-Up Plate. Change the pipette tips after each of the three corresponding rows of sample are pooled onto the Clean-Up Plate. Repeat these steps until all of the PCR products are pooled. Examine the three PCR product plates to be sure that the full volume of each well was transferred and that the plates are empty. The final volume in each well on the Clontech Clean-Up Plate should be approximately 320 μL. To avoid piercing the Clean-Up Plate membrane, do not pipette up and down in the plate, and do not touch the bottom of the plate. Be very careful when pooling the third set of PCR products, as the wells are very full. Avoid cross-contaminating neighboring wells with small droplets. Also, pipette very carefully to avoid the formation of air bubbles. Air bubbles will slow drying.

To purify the PCR products: Load the Clontech Clean-Up Plate with samples onto the manifold. Cover the plate to protect the samples from environmental contaminants. For example, the lid from a pipette tip box may be used. Turn on the vacuum and slowly bring it up to 600 mbar. Three water washes should be performed to properly purify the PCR products. Be sure to completely dry the membrane after the third wash. P1 P2 P3 CUP—BL Clean-Up Plate Transfer and pool each PCR product from plates P1, P2 and P3 to the corresponding well of the Clean-Up Plate. For example, transfer and pool the PCR product from well A1 of plates P1, P2 and P3 to the corresponding row and well on the Clean-Up Plate. P1, P2 and P3=PCR Product Plates=Pooled PCR product from row A of plate BL=bottom left. Check the vacuum by gently trying to lift the middle section of the manifold off the base. Be very careful not to lose any sample. You should not be able to lift the middle section off the base. Maintain the vacuum at 600 mbar until all of the wells are dry (approximately 1.5 to 2 hours). The vacuum regulator may sound like it is leaking. This sound is the pressure release working to limit the vacuum to 600 mbar. Wash the PCR products three times as follows, keeping the vacuum on the entire time: Add 75 mL ACCUGENE Water to a solution basin. Using a 12-channel P200 pipette, add 50 μL water to each well. Dry the wells (15 to 20 minutes). The top and bottom rows may take longer to filter and dry. D. Repeat steps B and C two additional times for a total of 3 water washes. After the third wash, tap the manifold firmly on the bench to force any drops on the sides of the wells to move to the bottom and be pulled through the plate. Allow the samples to dry completely. Drying after the third wash may take 45 to 75 minutes. Tilt and inspect the plate to confirm that the top and bottom rows are completely dry. For best results, do not allow the plate to sit on the manifold or the bench top for more than 90 minutes after the wells are completely dried. To prevent the dilution of DNA with water, ensure that every well is completely dry before adding RB Buffer.

To elute the PCR products: When the wells are completely dry after the third wash, turn off the vacuum. Carefully remove the plate from the manifold and immediately: A. Blot the bottom of the plate on a thick stack of clean absorbent paper to remove any remaining liquid. Dry the bottom of each well with an absorbent wipe. Aliquot 5 mL RB Buffer to a solution basin. Using a 12-channel P200 pipette, add 45 μL RB buffer to each well of the plate. Tightly seal the plate. Load the plate onto a Jitterbug plate shaker. Set the Jitterbug to setting 5 and moderately shake the plate for 10 minutes at room temperature. This setting (approximately 1000 rpm) allows as much movement as possible without losing liquid to the sides of the wells and film. Transfer 45 μL of each eluted sample from the Clontech Clean-Up Plate to the corresponding well of a fresh 96-well plate following these guidelines: Use a 12-channel P200 pipette set to 60 μL. Tilt the Clontech Clean-Up Plate at a 30 to 45 degree angle to move the liquid to one side of the well. Optional: use a plate support to keep the plate tilted at an angle (Well Plate Stand: Diversified Biotech, P/N WPST-1000). Pipette up and down 3 to 4 times before removing and transferring the eluate to a fresh 96-well reaction plate. Immediately blot the bottom of the plate and dry the bottom of each well. Any remaining liquid will quickly seep back into the wells. Go back into the well a second time and remove any remaining liquid. It is OK to touch the bottom of the filter. Do one of the following: Proceed immediately to the next step. If not proceeding immediately to the next step: A. Seal the plate with the eluted samples. B. Store the plate at −20° C.

Stage 6: Quantitation and Normalization. During this stage, three independent dilutions of each PCR product will be prepared in optical plates. The diluted PCR products are quantitated and the OD measurements from each plate are averaged. Once the concentration of each reaction is determined, normalize each reaction to 2 μg/μL in RB Buffer. The following equipment and consumables are required for this stage: 1 Plate of purified PCR product. The following reagents and equipment are used for this stage: 1 cooling chamber, double, chilled to 4° C. (do not freeze), 1 ice bucket, filled with ice, 1 marker, fine point, permanent, 1, single channel P20 pipette, 1 single channel P100 pipette, 1 single channel P1000 pipette, 1 12-channel P20 (accurate to within ±5%) pipette, 1 12-channel P200 pipette, full racks of tips for pipettes listed above; and 4 optical Plates, for example, the UV Star Transparent, 96-well. Use the optical plate recommended for use with your plate reader. Also used are 1 96-well reaction plate, 1 centrifuge plate, 5 Plate seals, 1 Spectrophotometer plate reader, 1 100 mL Solution basin, and 1 Vortexer. Prepare three independent dilutions of each sample for accurate concentration measurement. Average the results for each individual sample before normalizing. The sample in each well should be normalized to 2 μg/μL in RB Buffer (90 μg in 45 μL RB Buffer). For best results, do not determine an average concentration to use for every well. The amount of DNA added to the arrays has been optimized for the best performance. Since not all wells will contain the same amount of DNA after purification, the eluted PCR products should be carefully normalized to 2 μg/μL before continuing to Stage 7: Fragmentation. Normalize samples using RB Buffer (not water) to maintain the correct pH for subsequent steps. The accuracy of the OD measurement is critical. Carefully follow the steps below and be sure the OD measurement is within the quantitative linear range of the instrument (0.2 to 0.8 OD). The spectrophotometer plate reader should be calibrated regularly to ensure correct readings. This protocol has been optimized using a UV spectrophotometer plate reader for quantitation. NOTE: The NanoDrop will give different quantitation results. This protocol has not been optimized for use with this instrument. Reagents Required for Stage 6: Quantitation and Normalization: as needed RB Buffer (from Clontech DNA Amplification Clean-Up Kit) 75 mL ACCUGENE water, molecular biology-grade addition, the NanoDrop quantifies a single sample at a time and is not amenable to 96-well plate processing. When normalizing samples, be sure to use a 96-well plate that is compatible with the thermal cycler on which the 500K Fragment thermal cycler program will be run (Stage 7: Fragmentation). Turn on the Spectrophotometer Plate Reader Turn on the spectrophotometer now and allow it to warm for 10 minutes before use. To prepare the work area place a double cooling chamber on ice. Label the 96-well reaction plate Fragment as this plate will also be used for the next stage), and place on the cooling chamber. Place the following on the bench top: Optical plates, Solution basin and ACCUGENE water. Label each optical plate as follows: OP1, OP2, OP3, OP4. Vortex the RB Buffer and place on the bench top. Prepare the purified, eluted PCR product plate as follows: If the plate was frozen, allow it to thaw in a cooling chamber on ice. Vortex the center of the plate at high speed for 3 sec. Spin down the plate at 2000 rpm for 30 sec. Place the plate on the bench top.

To prepare three diluted aliquots of the purified samples: Pour 75 mL of room temperature ACCUGENE water into the solution basin. Using a 12-channel P200 pipette, aliquot 198 μL of water to: Each well of optical plates 1, 2 and 3 and the first four rows of optical plate 4. Using a 12-channel P20 pipette transfer 2 μL of each purified PCR product from rows A through G of the purified sample plate to the corresponding rows and wells of optical plates 1, 2 and 3. Pipette up and down 2 times after each transfer to ensure that all of the product is dispensed. Examine the pipette tips and aliquots before and after each dispense to ensure that exactly 2 μL has been transferred. Transfer 2 μL of each purified PCR product from row H of the purified sample plate to the corresponding rows and wells of optical plate 4. Again, pipette up and down 2 times after each transfer, and examine the pipette tips and aliquots before and after each dispense. The result is a 100-fold dilution. Two of the wells containing water only will serve as blanks. Set a 12-channel P200 pipette to 180 μL. Mix each sample by pipetting up and down 5 to 10 times. Be careful not to scratch the bottom of the plate, or to introduce air bubbles. Two of the wells on each optical plate should be set up as blanks containing ACCUGENE water only. The 12-channel P20 pipette should be accurate to within ±5%. Repeat this procedure and prepare three plates of diluted PCR product to test. Be sure to keep two wells as blanks (water only) on each plate.

To quantitate the diluted PCR product: measure the OD of each PCR product at 260, 280 and 320 nm. OD280 and OD320 are used as process controls. Their use is described under Process Control Metrics below. Determine the OD260 measurement for the water blank. Determine the concentration of each PCR product as follows: A. Take 3 OD readings for every sample (1 from each optical plate; P1, P2, P3 and P4). OD1=(sample OD)−(water blank OD) OD2=(sample OD)−(water blank OD) OD3=(sample OD)−(water blank OD) B. Average the 3 readings for each sample to obtain an Average Sample OD: Average Sample OD=(OD1+OD2+OD3)÷3 C. Calculate the undiluted sample concentration for each sample using the Average Sample OD: Sample concentration in μg/μL=Average Sample OD □ [symbol] (0.05 μg/μL) □ [symbol] 100 Apply the convention that 1 absorbance unit at 260 nm equals 50 μg/mL (equivalent to 0.05 μg/μL) for double-stranded PCR products. This convention assumes a path length of 1 cm. Consult your spectrophotometer handbook for further information. ASSESS THE OD READINGS Follow the guidelines below for assessing and troubleshooting OD readings. Average Sample OD A typical average sample OD is 0.5 to 0.7. This OD range is equivalent to a final PCR product concentration of 2.5 to 3.5 μg/μL. It is based on the use of a conventional UV spectrophotometer plate reader and assumes a path length of 1 cm. Process Control Metrics Evaluate the process control metrics as follows: the OD260/OD280 ratio should be between 1.8 and 2.0. For best results, do not proceed if this metric falls outside of this range. The OD320 measurement should be very close to zero (0±0.005). Troubleshooting: Average Sample OD is greater than 0.7 (3.5 μg/μL) If the average sample OD of three independent measurements is greater than 0.7 (calculated concentration greater than 3.5 μg/μL), a problem exists with either the elution of PCR products or the OD reading. The limit on PCR yield is approximately 3.5 μg/μL, as observed in practice and as predicted by the mass of dNTPs in the reaction. Possible causes include: The purified PCR product was eluted in a volume less than 45 μL. The purified PCR product was not mixed adequately before making the 1:100 dilution. The diluted PCR product was not mixed adequately before taking the OD reading. The water blank reading was not subtracted from each sample OD reading. The spectrophotometer plate reader may require calibration. Pipettes may require calibration. There may be air bubbles or dust in the OD plate. There may be defects in the plastic of the plate. The settings on the spectrophotometer plate reader or the software may be incorrect. OD calculations may be incorrect and should be checked. Reliance on any single OD reading may give an outlier result. Make three independent dilutions and take three independent OD readings per dilution. Troubleshooting: Average Sample OD is Less Than 0.5 (2.5 μg/μL) If the average sample OD of three independent measurements is less than 0.5 (calculated concentration less than 2.5 μg/μL), a problem exists with either the genomic DNA, the PCR reaction, the elution of purified PCR products, or the OD readings. Possible problems with input genomic DNA that would lead to reduced yield include: The presence of inhibitors (heme, EDTA, etc.). Severely degraded genomic DNA. Inaccurate concentration of genomic DNA. NOTE: Check the OD reading for the PCR products derived from RefDNA 103 as a control for these issues. To prevent problems with the PCR reaction that would lead to reduced yield: use the recommended reagents and vendors (including ACCUGENE water) for all PCR mix components. Thoroughly mix all components before making the PCR Master Mix. Pipette all reagents carefully, particularly the PCR Primer, when making the master mix. Check all volume calculations for making the master mix. Store all components and mixes on ice when working at the bench. For best results, do not allow reagents to sit at room temperature for extended periods of time. Be sure to use the recommended PCR plates. Plates from other vendors may not fit correctly in the thermal cycler block. Differences in plastic thickness and fit with the thermal cycler may lead to variance in temperatures and ramp times. Be sure to use the correct cycling mode when programming the thermal cycler (maximum mode on the GeneAmp PCR System 9700; calculated mode on the MJ Tetrad PTC-225). Be sure to use silver or gold-plated silver blocks on the GENEAMP PCR System 9700 (other blocks are not capable of maximum mode, which will affect ramp times). Use the recommended plate seal. Make sure the seal is tight and that no significant evaporation occurs during the PCR. NOTE: The Mapping 500K PCR reaction amplifies a size range of fragments that represents 15-20% of the genome. The Mapping 500K arrays are designed to detect the SNPs that are amplified in this complex fragment population. Subtle changes in the PCR conditions may not affect the PCR yield, but may shift the amplified size range up or down very slightly. This can lead to reduced amplification of SNPs that are assayed on the array set, subsequently leading to lower call rates. Troubleshooting Possible Problems with the Elution or OD Readings—possible causes include: The purified PCR product was eluted in a volume greater than 45 μL. The purified PCR product was not mixed adequately before making the 1:100 dilution. The diluted PCR product was not mixed adequately before taking the OD reading. The water blank reading was not subtracted from each sample OD reading. The spectrophotometer plate reader may require calibration. Pipettes may require calibration. There may be air bubbles or dust in the OD plate. There may be defects in the plastic of the plate. The settings on the spectrophotometer plate reader or the software may be incorrect. OD calculations may be incorrect and should be checked. Reliance on any single OD reading may give an outlier result. Make three independent dilutions and take three independent OD readings per dilution. Trouble shooting: problem: OD260/OD280 ratio is not between 1.8 and 2.0-possible causes include: The PCR product may be not be sufficiently purified. Be sure to perform three water washes and check to be sure the vacuum manifold is working properly. An error may have been made while taking the OD readings. Trouble shooting: problem: Average Sample OD is less than 0.5 (2.5 μg/μL). To normalize the samples: Calculate the volume of RB Buffer required to normalize each sample. Using a single-channel P20 pipette, add the calculated volume of RB Buffer to each well (the value of X). Using a single-channel P100 pipette, add the calculated volume of purified PCR product (the value of Y) to the corresponding well with RB Buffer. The total volume of each well is now 45 μL. After normalization, each well should contain 90 μg of purified PCR product in a volume of 45 μL (or 2 μg/μL). Seal the plate with adhesive film. Vortex the center of the plate at high speed for 3 sec. Trouble shooting: problem: The OD320 measurement is significantly larger than zero (0±0.005). Possible causes include: Precipitate may be present in the eluted samples. Be sure to add diluted EDTA to PCR products before purification. There may be defects in the OD plate. Air bubbles in the OD plate or in solutions. Formula X μL RB Buffer=45 μL−(Y μL purified PCR product) Where: Y=The volume of purified PCR product that contains 90 μg The value of Y is calculated as: Y μL purified PCR product=(90 μg)÷(Z μg/μL) Z=the concentration of purified PCR product in μg/μL Spin down the plate at 2000 rpm for 30 sec and place back in the cooling chamber. Proceed immediately to the next stage. Because the DNA concentration in each sample is different, the volume transferred to each well will differ. For optimal performance, it is critical that the contents of each well be normalized to 2 μg of DNA/μL before proceeding to the next step.

Stage 7: Fragmentation: During this stage the purified, normalized PCR products will be fragmented using Fragmentation Reagent. First dilute the Fragmentation Reagent by adding the appropriate amount of Fragmentation Buffer and ACCUGENE water. Quickly add the diluted reagent to each reaction, place the plate onto a thermal cycler, and run the 500K Fragment program. Once the program is finished, check the results of this stage by running 4 μL of each reaction on a 4% TBE gel. The input required from Stage 6: Quantitation and Normalization is: 1 Plate of quantitated, normalized PCR product in a cooling chamber on ice. For best results, use the PCR plate, adhesive film and thermal cyclers listed. Equipment and Consumables Required for Stage 7: Fragmentation: 1 Cooler, chilled to −20° C. 1 Cooling chamber, double, chilled to 4° C. (do not freeze) 1 Ice bucket, filled with ice, 1 Marker, fine point, permanent, 1 Microcentrifuge, 1 Pipette, single channel P20 1 Pipette, single channel P100 1 Pipette, single channel P1000, 1 Pipette, 12-channel P20 (accurate to within±5%), as needed Pipette tips for pipettes listed above; full racks 1 Plate centrifuge 1 Plate seal, 1 Thermal cycler, 2 Eppendorf 1.5 mL tubes, and 2 strips of 12 tubes cut from the Bio-Rad 96-well unskirted PCR plate, P/N MLP-9601. For this stage, the strip tubes should be cut from this particular plate and 1 vortexer. The amounts listed are sufficient to process one full 96-well reaction plate. The following gels and related materials are required for this stage. The amounts listed are sufficient to process one full 96-well reaction plate. Reagents Required for Stage 7: Fragmentation: 1 vial Fragmentation Buffer (10×), 1 vial Fragmentation Reagent (DNase I), 2 mL ACCUGENE water, molecular biology-grade. 5 4% TBE Gel 10 DNA Markers, 5 μL each as needed Gel loading solution

Purified PCR product should be normalized to 90 μg DNA in 45 μL RB Buffer. The degree of fragmentation is critical. Perform this stage carefully to ensure uniform, reproducible fragmentation. The Fragmentation Reagent is extremely temperature sensitive. It rapidly loses activity at higher temperatures. To avoid loss of activity:—Dilute the Fragmentation Reagent immediately prior to use.—Keep at −20° C. until ready to use. Transport and hold in a −20° C. cooler. Return to the cooler immediately after use.—Perform these steps rapidly and without interruption. The Fragmentation Reagent (DNase I) may adhere to the walls of some microfuge tubes and 96-well plates. To ensure the accurate amount of DNase I in the fragmentation reaction (Stage 7: Fragmentation), the strip tubes used for this stage should be cut from Bio-Rad 96-well unskirted PCR plates, P/N MLP-9601. The Fragmentation Reagent is viscous and requires extra care when pipetting. Follow these guidelines:—When aspirating, allow enough time for the correct volume of solution to enter the pipette tip.—Avoid excess solution on the outside of the pipette tip. Using in-house ddH2O or other water can negatively affect the results. The reaction in Stage 7: Fragmentation is particularly sensitive to pH and metal ion contamination. All additions, dilutions and mixing should be performed on ice. Thaw Reagents Thaw the Fragmentation Buffer (10×) on ice. To prepare the work area: Place a double cooling chamber and a cooler on ice. Place the ACCUGENE Water on ice. Prepare the Fragmentation Buffer as follows: Vortex 3 times, 1 sec each time. Pulse spin for 3 sec. Place the buffer in the cooling chamber on ice. Cut two strips of 12 tubes from a Bio-Rad 96-well unskirted PCR plate (P/N MLP-9601). Strip tubes should be cut from this particular plate. Label and place the following in the cooling chamber on ice: two strips of 12 tubes labeled Buffer and FR. One 1.5 mL Eppendorf tube labeled Frag MM. Plate of purified, normalized PCR product from the previous stage. Leave the Fragmentation Reagent at −20° C. until ready to use. Preheat the Thermal Cycler Block The block should be heated to 37° C. before samples are loaded. To preheat the thermal cycler: Power on the thermal cycler and preheat the block to 37° C. Allow it to heat for 10 minutes before loading samples. Add Fragmentation Buffer to Samples To prepare the samples for Fragmentation: Aliquot 50 μL of 10× Fragmentation Buffer to each tube of the 12-tube strip labeled Buffer. Using a 12-channel P20 pipette, add 5 μL of Fragmentation Buffer to each sample in the 96-well reaction plate. Check the pipette tips each time to ensure that all of the buffer has been dispensed. The total volume in each well is now 50 μL. Dilute the Fragmentation Reagent To dilute the Fragmentation Reagent: Read the Fragmentation Reagent tube label and record the concentration. All of the additions in this procedure should be performed on ice. The concentration of stock Fragmentation Reagent (U/μL) may vary from lot-to-lot. Therefore, read the label on the tube and record the stock concentration before diluting this reagent. Use the formula provided to accurately calculate the dilution required. If the concentration is 2 or 3 U/μL, dilute the Fragmentation Reagent using the volumes show. If the concentration is not 2 or 3 U/μL, use the formula below to calculate the dilution required to bring the reagent to a final concentration of 0.05 U/μL. Dilute the Fragmentation Reagent to 0.05 U/μL as follows or the dilution formula calculation: To the 1.5 mL Eppendorf tube on ice: 1) Add the ACCUGENE water and Fragmentation Buffer. Dilution Recipes for Fragmentation Reagent Concentrations of 2 or 3 U/μL: ACCUGENE water 525 μL or 530 μL, Fragmentation Buffer 60 μL or 60 μL, Fragmentation Reagent 15 μL or 10 μL. Total (enough for 96 samples) is 600 μL for both. Formula Y=0.05 U/μL 600 μL X U/μL Where: Y=number of μL of stock Fragmentation Reagent X=number of U of stock Fragmentation Reagent per μL (per label on tube) 0.05 U/μL=final concentration of Fragmentation Reagent 600 μL=final volume of diluted Fragmentation Reagent (enough for 96 reactions). Allow to cool on ice. Remove the Fragmentation Reagent from the freezer and: Immediately pulse spin for 3 sec. Spinning is required because the Fragmentation Reagent tends to cling to the top of the tube, making it warm quicker. Immediately place in a cooler. Add the Fragmentation Reagent to the 1.5 mL Eppendorf tube. Vortex the diluted Fragmentation Reagent at high speed 3 times, 1 sec each time. Pulse spin for 3 sec and immediately place on ice. Proceed immediately to the next set of steps, Add Diluted Fragmentation Reagent to the Samples. Add Diluted Fragmentation Reagent to the Samples To add diluted Fragmentation Reagent to the samples: Quickly and on ice, aliquot 50 μL of diluted Fragmentation Reagent to each tube of the 12 tube strip labeled FR. Using a 12-channel P20 pipette, add 5 μL of diluted Fragmentation Reagent to each sample. For best results, do not pipette up and down. Seal the plate and inspect the edges to ensure that it is tightly sealed. Reagent Volume/Sample Sample with Fragmentation Buffer 50 μL Diluted Fragmentation Reagent (0.05 U/μL) 5 μL Total 55 μL. Vortex the center of the plate at high speed for 3 sec. Place the plate in a chilled plastic plate holder and spin it down at 4° C. at 2000 rpm for 30 sec. Immediately load the plate onto the pre-heated block of the thermal cycler (37° C.) and run the 500K Fragment program. Discard any remaining diluted Fragmentation Reagent. Diluted Fragmentation Reagent should not be reused. Proceed directly to the next stage. Concurrently, check the fragmentation reaction by running gels as described below. To minimize solution loss due to evaporation, make sure that the plate is tightly sealed prior to loading onto the thermal cycler. The MJ thermal cyclers are more prone to evaporation. 500K Fragment Program Temperature Time 37° C. 35 minutes 95° C. 15 minutes 4° C. Hold. To ensure that fragmentation was successful: When the 500K Fragment program is finished: Remove the plate from the thermal cycler. Spin down the plate at 2000 rpm for 30 sec, and place in a cooling chamber on ice. Dilute 4 μL of each fragmented PCR product with 4 μL gel loading dye. Run on 4% TBE gel with the BioNexus All Purpose Hi-Lo ladder at 120V for 30 minutes to 1 hour. Inspect the gel. Average fragment size is <180 bp.

Stage 8: Labeling. During this stage, the fragmented samples will be labeled using the GENECHIP DNA Labeling Reagent. Prepare the Labeling Master Mix, add the mix to each sample, place the samples onto a thermal cycler and run the 500K Label program. The following equipment and consumables are required for this stage: 1 Plate of fragmented DNA. Use only the PCR plate, adhesive film and thermal cyclers listed in Equipment and Consumables Required for Stage 8. 1 Cooler, chilled to −20° C., 1 Cooling chamber, double, chilled to 4° C. (do not freeze), 1 Ice bucket, filled with ice, 1 Marker, fine point, permanent, 1 Microcentrifuge, 1 Pipette, single channel P200, 1 Pipette, single channel P1000, 1 Pipette, 12-channel P20 (accurate to within ±5%), as needed Pipette tips for pipettes listed above in full racks, 1 Plate centrifuge, 1 Plate seal, 1 Thermal cycler, 1 15 mL centrifuge tube, 1 strip of 12 tubes, and 1 Vortexer The following reagents are required for this stage. The amounts listed are sufficient to process one full 96-well reaction plate. To minimize sample loss due to evaporation, tightly seal the plate before running the 500K Label thermal cycler program. Thaw Reagents Thaw the following reagents on ice: 1 vial GENECHIP DNA Labeling Reagent (30 mM), 1 vial Terminal Deoxynucleotidyl Transferase (TdT; 30 U/μL), and 2 vials Terminal Deoxynucleotidyl Transferase Buffer (TdT Buffer; 5×). Leave the TdT enzyme at −20° C. until ready to use. Place a double cooling chamber and a cooler on ice. Prepare the reagents as follows: vortex each reagent at high speed 3 times, 1 sec each time. Pulse spin for 3 sec. C. Place in the cooling chamber. Label and place the following in the cooling chamber: One strip of 12 tubes labeled MM One 15 mL centrifuge tube labeled MM, and the plate of fragmented reactions from the previous stage. The thermal cycler block should be heated to 37° C. before samples are loaded. Keep all reagents and tubes on ice while preparing the Labeling Master Mix. To prepare the Labeling Master Mix: Add the following to the 15 mL centrifuge tube on ice using the volumes listed. 5× TdT Buffer GENECHIP DNA Labeling Reagent. Remove the TdT enzyme from the freezer and immediately place in the cooler. Pulse spin the enzyme for 3 sec; then immediately place back in the cooler. Add the TdT enzyme to the master mix. Vortex the master mix at high speed 3 times, 1 sec each time. Pulse spin for 3 sec. Immediately proceed to the next set of steps. To add the Labeling Master Mix to the samples: Keep samples in the cooling chamber and all tubes on ice when making additions. Aliquot 178 μL of Labeling Master Mix to each tube of the strip tubes. Add the Labeling Master Mix as follows: using a 12-channel P20 pipette, aliquot 19.5 μL of Labeling Master Mix to each sample. Pipette up and down one time to ensure that all of the mix is added to the samples. The total volume in each well is now 70 μL. Labeling Master Mix for 1 Sample or for 96 Samples (15% extra): TdT Buffer (5×) 14 μL or 1545.6 μL, GENECHIP DNA Labeling Reagent (30 mM) 2 μL or 220.8 μL, TdT enzyme (30 U/μL) 3.5 μL or 386.4 μL. Seal the plate tightly with adhesive film. Vortex the center of the plate at high speed for 3 sec. Spin down the plate at 2000 rpm for 30 sec. Place the plate on the pre-heated thermal cycler block, and run the 500K Label program. Samples can remain at 4° C. overnight. When the 500K labeling program is finished: remove the plate from the thermal cycler. Spin down the plate at 2000 rpm for 30 sec. Reagent Volume/Rx Fragmented DNA (less the 4 μL used for gel analysis) 50.5 μL Labeling Mix 19.5 μL Total 70 μL. Check to ensure that the plate is tightly sealed to minimize evaporation while on the thermal cycler, particularly around the wells on the edge of the plate. 500K Label Program is 37° C. for 4 hours, 95° C. for 15 minutes and hold at 4° C. Samples can remain at 4° C. overnight. Either proceed to the next stage or freeze the samples at −20° C.

Stage 9: Target Hybridization ABOUT THIS STAGE During this stage, each sample is loaded onto either a GENECHIP Human Mapping 250K Sty Array or a 250K Nsp Array. Three methods for performing this stage are presented. Method 1—Using a GeneAmp® PCR System 9700 Requires the use of a GENEAMP PCR System 9700 thermal cycler located cycler adjacent to the hybridization ovens. Samples are on a 96-well reaction plate. Method 2—Using an Applied Biosystems 2720 Thermal Cycler or an MJ Tetrad PTC-225 Thermal Cycler. Requires the use of an Applied Biosystems 2720 Thermal Cycler or an MJ Tetrad PTC-225 Thermal Cycler located adjacent to the hybridization ovens. Samples are on a 96-well reaction plate. Method 3—Using Heat Blocks Requires the use of two heat blocks and Eppendorf tubes, one per sample. First, prepare a Hybridization Master Mix and add the mix to each sample. Then, based on the method you are using, denature the samples on a thermal cycler (methods 1 and 2) or on a heat block (method 3). After denaturation, load each sample onto the appropriate GENECHIP Human Mapping 250K Array (Nsp or Sty)—one sample per array. The arrays are then placed into a hybridization oven that has been preheated to 49° C. Samples are left to hybridize for 16 to 18 hours. Two operators are required for all of the methods. Location is main lab and ands-on time is approximately 2 hours Hybridization time is 16 to 18 hours. The following equipment and consumables are required for this stage. 1 Plate of labeled DNA. Equipment and consumables required for Stage 9: Target Hybridization using a thermal cycler: 1 cooling chamber, chilled to 4° C. (do not freeze), 96 GENECHIP 500K Arrays (one array per sample) 1 GENECHIP Hybridization Oven 640 1 Ice bucket, filled with ice 1 Pipette, single channel P200 1 Pipette, single channel P1000 As needed Pipette tips for pipettes listed above; full racks 1 Plate, Bio-Rad 96-well, P/N MLP-9601 1 Plate centrifuge, 2 Plate holders, centrifuge 1 Plate seal, 1 55 mL Solution basin, 1 Thermal cycler, 2 TOUGH-SPOTS per array, 1 centrifuge tube 50 mL, and 1 Vortexer. Hybridizing Samples Using Heat Blocks. Equipment and Consumables Required for Stage 9: Target Hybridization Using Heat Blocks: 1 Cooling chamber, chilled to 4° C. (do not freeze), 96 GENECHIP Human Mapping 250K Sty Arrays or GENECHIP Human Mapping 250K Nsp Arrays, one array per sample is required, 1 GENECHIP Hybridization Oven 640, 2 Heat blocks, 1 Ice bucket, filled with ice, 1 single channel P200 pipette, 1 single channel P1000 pipette and full racks of pipette tips for pipettes listed above; 1 Plate centrifuge 1 Plate seal, 1 55 mL Solution basin, 4 Timers, 1 50 mL centrifuge tube, 96 1.5 mL EPPENDORF Safe-Lock tubes, (one tube per sample), 2 TOUGH-SPOTS per array and 1 Vortexer. The following reagents are required for this stage. The amounts listed are sufficient to process one full 96-well reaction plate. To help ensure the best results, carefully read the information below before beginning this stage of the protocol. This procedure requires two operators working simultaneously when loading samples onto arrays and placing arrays in the hybridization ovens. If using a thermal cycler, it is critical that the samples remain at 49° C. after denaturation and while being loaded onto arrays.

Reagents Required for Stage 9: Target Hybridization: 5 mL (1 tube) Denhart's Solution, 1.5 mL (1 tube) DMSO, 0.5 mL (1 vial) EDTA, 1 mL (1 vial) Herring Sperm DNA (HSDNA), 500 μL (1 vial) Human Cot-1 DNA, 80 g MES Hydrate SigmaUltra, 200 g MES Sodium Salt, 16 mL (1 tube) Tetramethyl Ammonium Chloride (TMACL; 5M), 10 mL (1 vial) Tween-20, 10%, and 250 μL (1 vial) Oligo Control Reagent (OCR). When adding to the Hybridization Master Mix, pipette DMSO into the middle of the tube. For best results, do not touch the sides of the tube as the DMSO can leach particles out of the plastic which, in turn, may cause high background. DMSO is light sensitive and should be stored in a dark glass bottle. For best results, do not store in a plastic container and be sure to allow the arrays to equilibrate to room temperature; otherwise, the rubber septa may crack and the array may leak. An accurate hybridization temperature is critical for this assay. Therefore, we recommend that the hybridization ovens be serviced at least once per year to ensure that they are operating within manufacture specifications. Gloves, safety glasses, and lab coats should be worn when preparing the Hybridization Master Mix. Prepare a 12× MES Stock Solution The 12× MES stock solution can be prepared in bulk and kept for at least one month if properly stored. Proper storage: Protect from light using aluminum foil Keep at 4° C. To prepare 1000 mL of 12× MES Stock Solution: (1.25 M MES, 0.89 M [Na+]), combine: 70.4 g MES hydrate 193.3 g MES sodium salt 800 mL ACCUGENE water. Mix and adjust volume to 1,000 mL. The pH should be between 6.5 and 6.7. Filter the solution through a 0.2 μm filter. Protect from light using aluminum foil and store at 4° C. Preheat the Hybridization Ovens To preheat the hybridization ovens: Turn each oven on and set the temperature to 49° C. Set the rpm to 60. Turn the rotation on and allow to preheat for 1 hour before loading arrays. Do not autoclave. Store between 2° C. and 8° C., and shield from light using aluminum foil. Discard solution if it turns yellow. Thaw Reagents If the labeled samples from the previous stage were frozen: Thaw the plate on the bench top. Vortex the center of the plate at high speed for 3 sec. Spin down the plate at 2000 rpm for 30 sec. Place in a cooling chamber on ice. If hybridizing samples using Method 1 or 2, the labeled samples should be placed in a Bio-Rad unskirted 96-well plate (P/N MLP-9601). For Method 2, the plate will be cut into 4 strips of 24 wells each. Preheat the Thermal Cycler Lid A thermal cycler is required only if hybridizing samples using Method 1 or 2. Power on the thermal cycler to preheat the lid. Leave the block at room temperature. Heat blocks are required only if hybridizing samples using Method 3. To prepare the heat blocks preheat one to 99° C. and the other to 49° C. An accurate hybridization temperature is important for this assay. To prepare the arrays: unwrap the arrays and place on the bench top, septa-side up. Mark each array with a meaningful designation (e.g., a number) to ensure that you know which sample is loaded onto each array. Insert a 200 μL pipette tip into the upper right septum of each array. Allow the arrays to warm to room temperature by leaving on the bench top 10 to 15 minutes. As an option, you can prepare a larger volume of Hybridization Master Mix than required. The extra mix can be aliquoted and stored at −20° C. for up to one week. To prepare the Hybridization Master Mix add the reagents to the 50 mL centrifuge tube in the order listed. Mix well. If making a larger volume, aliquot out 20.9 mL, and store the remainder at −20° C. for up to one week. To ensure that the data collected during scanning is associated with the correct sample, number the arrays in a meaningful way. To prepare stored Hybridization Master Mix: Place the stored Hybridization Master Mix on the bench top, and allow to warm to room temperature. Vortex at high speed until the mixture is homogeneous and without precipitates (up to 5 minutes). Pulse spin for 3 sec. Hybridization Master Mix: for 1 Array or 96 Arrays with (15% extra): MES (12×; 1.25 M) 12 μL or 1320 μL, Denhardt's Solution (50×) 13 μL or 1430 μL, EDTA (0.5 M) 3 μL or 330 μL, HSDNA (10 mg/mL) 3 μL or 330 μL, OCR, 0100 2 μL or 220 μL, Human Cot-1 DNA (1 mg/mL) 3 μL or 330 μL, Tween-20 (3%) 1 μL or 110 μL, DMSO (100%) 13 μL or 1430 μL, and TMACL (5 M) 140 μL or 1540 mL, for a total of 190 μL per array or 20.9 mL for 96 arrays.

Method 1—Using a GENEAMP PCR System 9700 The thermal cycler used for this method should be a GENEAMP PCR System 9700 located adjacent to the hybridization ovens. This particular thermal cycler is recommended because of the way the lid operates. You can slide it back one row at a time as samples are loaded onto arrays. Keeping the remaining rows covered prevents condensation in the wells. To add Hybridization Master Mix and denature the samples: Pour 20.9 mL Hybridization Master Mix into a solution basin. Using a 12-channel P200 pipette, add 190 μL of Hybridization Master Mix to each sample on the Label Plate. Total volume in each well is 260 μL. Seal the plate tightly with adhesive film. Vortex the center of the plate for 3 minutes. Spin down the plate at 2000 rpm for 30 sec. Cut the adhesive film between each row of samples. Do not remove the film. Place the plate onto the thermal cycler and close the lid. Run the 500K Hyb program. 500K Hyb Program: 95° C. 10 minutes and 49° C. Hold.

Load the Samples onto Arrays. This procedure uses 2 operators working simultaneously. Operator 1 loads the samples onto the arrays; Operator 2 covers the septa with TOUGH-SPOTS and loads the arrays into the hybridization ovens. To load the samples onto arrays: Operator 1: When the plate reaches 49° C., slide back the lid on the thermal cycler enough to expose the first row of samples only. Remove the film from the first row. Using a single-channel P200 pipette, remove 200 μL of denatured sample from the first well. Immediately inject the sample into an array. Pass the array to Operator 2. Remove 200 μL of sample from the next well and immediately inject it into an array. Pass the array to Operator 2. Repeat this process one sample at a time until the entire row is loaded. Place a fresh strip of adhesive film over the completed row. Slide the thermal cycler lid back to expose the next row of samples. Repeat steps 3 through 10 until all of the samples have been loaded onto arrays. Operator 2: Cover the septa on each array with a Tough-Spot. When 4 arrays are loaded and the septa are covered: Load the arrays into an oven tray evenly spaced. Immediately place the tray into the hybridization oven. For best results, do not allow loaded arrays to sit at room temperature for more than approximately 1 minute. Ensure that the oven is balanced as the trays are loaded, and ensure that the trays are rotating at 60 rpm at all times. Because you are loading 4 arrays per tray, each hybridization oven will have a total of 32 arrays. Operators 1 and 2: Load no more than 32 arrays in one hybridization oven at a time. All 96 samples should be loaded within 1 hour. Store the remaining samples and any samples not yet hybridized in a tightly sealed plate at −20° C. Allow the arrays to rotate at 49° C., 60 rpm for 16 to 18 hours. This temperature is optimized for this product.

Method 2—Using an Applied Biosystems 2720 Thermal Cycler or an MJ Tetrad PTC-225 Thermal Cycler For this method, you can use an Applied Biosystems 2720 Thermal Cycler or an MJ Tetrad PTC-225 Thermal Cycler. The thermal cycler should be located adjacent to the hybridization ovens. Because the lids on these thermal cyclers do not slide back, you will process 24 samples at a time. Add Hybridization Master Mix and Denature To add Hybridization Master Mix and denature the samples: Pour 20.9 mL Hybridization Master Mix into a solution basin. Using a 12-channel P200 pipette, add 190 μL of Hybridization Master Mix to each sample on the Label Plate. Total volume in each well is 260 μL. Seal the plate tightly with adhesive film. Vortex the center of the plate for 3 minutes. 5 Cut the plate into 4 strips of two rows each. Put each strip of 24 samples into a plate holder, 2 strips per holder. Spin down the strips at 2000 rpm for 30 sec. Cut the adhesive film between each row of samples. Do not remove the film. Place one set of 24 wells onto the thermal cycler and close the lid. Keep the remaining sets of wells in a cooling chamber on ice. Run the 500K Hyb program. Load the Samples onto Arrays This procedure requires 2 operators working simultaneously. Operator 1 loads the samples onto the arrays; Operator 2 covers the septa with TOUGH-SPOTS and loads the arrays into the hybridization ovens. To load the samples onto arrays: Operator 1 When the plate reaches 49° C., open the lid on the thermal cycler. Remove the film from the first row. Using a single-channel P200 pipette, remove 200 μL of denatured sample from the first well. Immediately inject the sample into an array. Pass the array to Operator 2. Remove 200 μL of denatured sample and immediately inject it into an array. Pass the array to Operator 2. Repeat this process one sample at a time until all 24 samples are loaded onto arrays. 500K Hyb Program: 95° C. 10 minutes 49° C. Hold. Cover the wells with a fresh strip of adhesive film and place in the cooling chamber on ice. Remove the next strip of 24 wells and place it on the thermal cycler. Run the 500K Hyb program. Repeat steps 1 through 11 until all of the samples have been loaded onto arrays. Operator 2: Cover the septa on each array with a Tough-Spot. When 4 arrays are loaded and the septa are covered: Load the arrays into an oven tray evenly spaced. Immediately place the tray into the hybridization oven. For best results, do not allow loaded arrays to sit at room temperature for more than approximately 1 minute. Ensure that the oven is balanced as the trays are loaded, and ensure that the trays are rotating at 60 rpm at all times. Because you are loading 4 arrays per tray, each hybridization oven will have a total of 32 arrays. Operators 1 and 2: Load no more than 32 arrays in one hybridization oven at a time. All 96 samples should be loaded within 1 hour. Store the remaining samples and any samples not yet hybridized in a tightly sealed plate at −20° C. Allow the arrays to rotate at 49° C., 60 rpm for 16 to 18 hours.

Method-3. The following instructions require 2 operators working simultaneously, each processing two samples at a time. Batches of sixteen samples at a time are denatured and loaded onto arrays. Two heat blocks are required: one set to 99° C.; the other set to 49° C. Load Samples Onto a Heat Block. If the heat blocks are not turned on, preheat them now (set one to 99° C.; the other to 49° C.). Add 190 μL of Hybridization Master Mix to each 1.5 mL Eppendorf Safe-Lock tube. Transfer the labeled sample from the reaction plate to a tube containing Hybridization Master Mix (one sample per tube). The total volume is now 260 μL. Vortex at high speed 3 times, 1 sec each time. Pulse spin for 3 sec. Do one of the following: If denaturing and loading samples onto arrays now, place the tubes on ice. If not proceeding to denature and hybridization at this time, store the samples at −20° C. (the mix will not freeze). Place the tubes in batches of 16 at a time onto a heat block as follows: Reagent Volume/Sample Hybridization Master Mix 190 μL Labeled DNA 70 μL Total 260 ρL. Place four tubes onto a heat block at 99° C. and set a timer for 10 minutes. Wait 3 to 4 minutes, then place another 4 tubes onto the heat block and set another timer for 10 minutes. Repeat this procedure until there are 16 samples loaded onto the heat block. Remove Samples from heat block and load onto arrays. Two operators will perform this procedure at the same time, two samples per person. To load samples onto arrays, 16 samples at a time: When the first timer indicates 10 minutes has transpired: immediately remove the first samples (two per operator). Cool on crushed ice for 10 sec, then remove immediately. Pulse spin for 3 sec. Place the tubes back on the heat block at 49° C. for 1 minute. Remove tubes from the heat block, and check for precipitate. Using a single-channel P200 pipette, remove 200 μL of denatured sample from one tube. Immediately inject the sample into an array. Cover each septa with a Tough-Spot. Repeat steps 5 through 7 for the next sample. Immediately load the arrays into a hybridization oven tray, 4 arrays per tray evenly spaced. Cool for 10 sec only. If left on ice longer, aggregates may form. These aggregates will not break apart at 49° C. and will reduce your call rate. Cooling on ice is required for this method only due to the loose fit of the tubes on the heat blocks. This step helps to ensure that the samples cool quickly to 49° C.

### CONCLUSION

A method for detection of greater than about 500,000 Single Nucleotide Polymorphisms (SNPs) in samples of genomic DNA is disclosed. It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

We Claim:

1. A method for determining the genotype of more than 200,000 SNPs in a nucleic acid sample comprising:
(a.) obtaining a nucleic acid sample;
(b.) fragmenting said nucleic acid in a first fragmentation step to produce fragments;
(c.) ligating an adaptor to at least some of the fragments from step (b) to generate adapter-ligated fragments;
(d.) amplifying at least some of the adapter-ligated fragments from step (c) to obtain amplified fragments;
(e.) fragmenting the amplified fragments of step (d) in a second fragmentation step to produce sub-fragments;
(f.) labeling the sub-fragments;
(g.) hybridizing the labeled sub-fragments from step (f) to an array of probes, wherein said array comprises probes to interrogate the genotype of more than 200,000 different single nucleotide polymorphisms; and
(h.) detecting a hybridization pattern; and
(i.) analyzing the hybridization pattern to determime the genotype of at least 500,000 single nucleotide polymorphisms in said sample.

2. The method of claim 1, wherein said nucleic acid sample comprises genomic DNA or an amplification product of genomic DNA.

3. The method of claim 1, wherein said nucleic acid sample comprises cDNA or RNA.

4. The method of claim 1, wherein the more than 200,000 single nucleotide polymorphisms have an average minor allele frequency greater than 15% in a population.

5. The method of claim 1, wherein said first fragmentation step comprises fragmenting by a restriction enzyme.

6. The method of claim 5, wherein the restriction enzymes includes at least one variable nucleotide position in the enzyme recognition site.

7. A method of claim 5, wherein said restriction enzyme is selected from the group consisting of Nsp I and Sty I.

8. The method of claim 1 wherein different adaptor sequences are ligated to the different overhangs so that the ends are not self complementary.

9. A method for determining the genotype of more than 400,000 different single nucleotide polymorphisms in a nucleic acid sample comprising:
(a.) obtaining a nucleic acid sample and dividing the sample into a first aliquot and a second aliquot;
(b.) fragmenting said first aliquot and said second aliquot in a first fragmentation step to produce fragments, wherein said first aliquot is fragmented with a first restriction enzyme and said second aliquot is fragmented with a second restriction enzyme;
(c.) ligating an adaptor to at least some of the fragments from step (b) to generate adapter-ligated fragments;
(d.) amplifying at least some of the adapter-ligated fragments from step (c) to obtain amplified fragments;
(e.) fragmenting the amplified fragments of step (d) in a second fragmentation step to produce sub-fragments;
(f.) labeling the sub-fragments;
(g.) hybridizing the labeled sub-fragments from step (f) to a first and a second array of probes, wherein the labeled sub-fragments from said first aliquot are hybridized to said first array and the labeled sub-fragments from said second array are hybridized to said second array and wherein said first array comprises probes to interrogate the genotype of a first collection of more than 200,000 different single nucleotide polymorphisms and said second array comprises probes to interrogate the genotype of a second collection of more than 200,000 different single nucleotide polymorphisms and wherein the polymorphisms in the first collection are all different from the polymorphisms in the second collection; and
(h.) detecting a hybridization pattern; and
(i.) analyzing the hybridization pattern to determime the genotype of at least 400,000 different single nucleotide polymorphisms in said sample.

10. The method of claim 9 wherein the first restriction enzyme is Nsp I and the second restriction enzyme is Sty I.

11. The method of claim 1, wherein said amplification is done with a thermal stable polymerase.

12. The method of claim 1, wherein said thermal stable polymerase is a Taq polymerase with a N-terminal mutation that inactivates the 5′ exonuclease activity of Taq.

13. The method of claim 1, wherein said labeling is done with Terminal Deoxynucleotidyl Transferase.

14. The method of claim 1 wherein uracil is incorporated into the PCR amplification and the fragmentation is by incubation with a uracil DNA glycosidase and an AP endonuclease.

15. A kit comprising SEQ ID NOS 1074931 and 1074933.

16. The kit of claim 15 further comprising SEQ ID NOS: 1074932 and 1074934.

17. The kit of claim 15 wherein SEQ ID NOS: 1074931 and 1074933 are included as a mixture in a single tube.

18. The kit of claim 16 wherein SEQ ID NOS: 1074932 and 1074934 are included as a mixture in a single tube.

19. The kit of claim 16 further comprising a ligase and a ligase buffer.

20. The kit of claim 19 further comprising dNTPs and a buffer for PCR.

21. The kit of claim 20 further comprising a DNA polymerase.

22. The kit of claim 21 wherein the DNA polymerase is a thermal stable DNA polymerase.

23. The kit of claim 21 wherein the DNA polymerase is a Taq DNA polymerase with an N-terminal mutation that inactivates the 5′ exonuclease activity of Taq.

24. The kit of claim 21 wherein the DNA polymerase is selected from the group consisting of PLATINIM Taq, TITANIUM Taq and AMPLITAQ GOLD.

25. The kit of claim 21 wherein the DNA polymerase activity includes a heat inactivatable activity that inhibits the polymerase activity.

26. The kit of claim 20 further comprising Betaine.

27. The kit of claim 20 further comprising an array comprising a plurality of 25 nucleotide probes wherein each probe is 25 nucleotides of a sequence from SEQ ID NO: 1-1,074,930 and wherein there are at least 800,000 different probes each corresponding to a different sequence from SEQ ID NO: 1-1,074,930.

28. An array of probes for interrogating the genotype of more than 400,000 different human single nucleotide polymorphisms, that array comprising at least 400,000 different probe sets, wherein a probe set comprises at least a first and a second probe, wherein said first probe is at least 20 bases and is perfectly complementary to a first allele of a human single nucleotide polymorphism and said second probe is at least 20 bases and is perfectly complementary to a second allele of said human single nucleotide polymorphism; and wherein each probe on the array is 20 contiguous bases of a sequence from SEQ ID NO: 1-1,074,930 or its complement.

29. A collection of probes for interrogating the genotype of a plurality of at least 400,000 human single nucleotide polymorphisms distributed throughout the human genome; said collection of probes comprising a probe comprising at least 17 contiguous bases from each of at least 400,000 sequences from SEQ ID NO: 1-1,074,930 or the complements of SEQ ID NO: 1-1,074,930.

30. The collection of probes of claim 29 wherein each different probe sequence is attached to a solid support in a known or determinable location.

31. The collection of probes of claim 30 wherein the solid support is selected from the group consisting of a bead and a glass substrate.

32. The collection of probes of claim 29 wherein said collection of probes comprises a probe comprising at least 17 contiguous bases from each of at least 800,000 sequences from SEQ ID NO: 1-1,074,930 or the complements of SEQ ID NO: 1-1,074,930.

33. The array of claim 28 wherein each single nucleotide polymorphism39 is interrogated by at least 6 perfect match probes.

34. The array of claim 28 wherein the array comprises two distinct solid supports, each having probe sets to interrogate each of at least 200,000 human single nucleotide polymorphisms.

35. The array of claim 28 wherein the array comprises a first array and a second array, wherein the first array interrogates single nucleotide polymorphisms that are on fragments that are 200 to 2000 basepairs when the genome is digested with a first enzyme and the second array interrogates single nucleotide polymorphisms that are on fragments that are 200 to 2000 basepairs when the genome is digested with a second enzyme.

36. The array of claim 35 wherein the first enzyme is NspI and the second enzyme is StyI.
